(19) United States
(12) Patent Application Publication
(10) Pub. No.: US 2026/0120826 A1
(43) Pub. Date: Apr. 30, 2026

(54) SYSTEMS AND METHODS FOR GENERATING CLINICAL SUMMARIES

(71) Applicant: Oracle International Corporation, Redwood Shores, CA (US)

(72) Inventors: Caleb Joseph Frerking, Kansas City, MO (US); Mason Grosko, Lee’s Summit, MO (US); Landis James Duff, Overland Park, KS (US); Joseph James O’Malley, Mission, KS (US); Andrew Roberts, Overland Park, KS (US); Vadim Khotilovich, Leawood, KS (US); Eugene Florintsev, Austin, TX (US)

(73) Assignee: Oracle International Corporation, Redwood Shores, CA (US)

(21) Appl. No.: 19/368,660

(22) Filed: Oct. 24, 2025

Related U.S. Application Data
(60) Provisional application No. 63/712,370, filed on Oct. 25, 2024.

(51) Int. Cl. G16H 10/60

(52) U.S. Cl. CPC G16H 10/60

(57) ABSTRACT

A computer-implemented method includes receiving a query in natural language, generating an input for a generative large language model, the input including a prompt generated based on the query, and identifying a plurality of slots associated with a plurality of sections of a content item. The method further includes generating a query result based on the input, the query result including a subset of the plurality of slots selected, extracting one or more document chunks from a database storing a plurality of document chunks as one or more relevant document chunks associated with the query result, formatting the relevant document chunks into a response to the query, and providing the response to a client system. The plurality of document chunks is generated by dividing each content item of a plurality of content items into the plurality of document chunks based on sections within each content item.

### CROSS-REFERENCE TO RELATED APPLICATIONS

The present application is a non-provisional application of and claims the benefit and priority to U.S. Provisional Application No. 63/712,370, filed Oct. 25, 2024, the entire contents of which is incorporated herein by reference for all purposes.

### BACKGROUND

Traditional healthcare systems involving computer-based assistants often rely on retrieving information from multiple data sources such as electronic health records and other data sources. Often these data sources code this data using custom coding schemes. As a result, retrieving and formatting retrieved information for standardized assistant user interfaces can be challenging. Traditional Electronic Health Record (EHR) systems can also have complex interfaces that may be difficult to navigate and cumbersome to operate. When the process for retrieving or recording necessary patient information is inefficient, it can disrupt the natural flow of the patient interaction.

Solutions addressing these changes and others would be desirable.

### BRIEF SUMMARY

Techniques disclosed herein pertain to generative artificial intelligence (AI) systems, and, more specifically, to summary generation techniques using agentic AI systems.

In embodiments, a computer-implemented method includes receiving a first request from a client system to provide a summary information for a patient of a healthcare provider, the summary information comprising patient history information describing at least one of a medical condition and a medical history of the patient, determining at least one of a plurality of visit categories and a plurality of medical complaints for the patient, retrieving data relevant to the first request from a plurality of sources based the at least one of the plurality of visit categories and the plurality of medical complaints, generating a first input for a generative machine learning model, the first input comprising a first prompt based on the first request, generating, by the generative machine learning model, a first query result from the first input, the first query result comprising a narrative section and a structured section extracted from one or more portions of the data, generating, by the generative machine learning model, at least one suggested follow-on query selectable as a further request, formatting the first query result and the suggested follow-on query into a first brief summary, and providing the first brief summary to a user interface at the client system.

In certain embodiments, retrieving the data includes accessing an electronic health record (EHR) to retrieve records including at least one of medical history, records from prior medical visits, laboratory results, diagnostic results, past abnormal observation, previously noted chief complaints, known allergies, medication history, immunization records, insurance information, social history, family history, previous recommendations, past and future appointments, and messages sent through the EHR. In embodiments, accessing the EHR includes limiting a range of the records to items created within a predetermined time period prior to receiving the request. In embodiments, the method further includes updating the data relevant to the request based on updated information received from the patient at a visit to generate updated data, the updated information including at least one of vitals, one or more chief complaints for the visit, and intake notes at the visit. In embodiments, the method further includes receiving a second request from the client device based on the first brief summary, retrieving data relevant to the second request, the data relevant to the second request including the updated data, generating a second input for the generative machine learning model, the second input comprising a second prompt and one or more portions of the data relevant to the second request, generating, by the generative machine learning model, a second query result from the second input based on the retrieved data relevant to the second request, formatting the second query result into a second brief summary, and providing the second brief summary to the user interface at the client system.

In certain embodiments, the narrative section includes a first selection from the first query result arranged into natural language text, and the structured section includes a second selection from the first query result used to populate a plurality of preset windows within the first brief summary. In embodiments, at least one of the narrative section and the structured section includes one of a Uniform Resource Locator (URL) and a hovering window to enable display of additional information regarding a specific portion of the first brief summary.

In embodiments, a system includes a computer system comprising one or more processors and memory storing computer executable instructions that, when executed by the one or more processors, configure the computer system to at least receive a first request from a client system to provide a summary information for a patient of a healthcare provider, the summary information comprising patient history information describing at least one of a medical condition and a medical history of the patient, determine at least one of a category of visit and a plurality of medical complaints for the patient, the category including acute, wellness, follow-up, and generic, obtain information regarding a chief complaint for the visit, retrieve data relevant to the first request from a plurality of sources based on the at least one of the category of visit and the plurality of medical complaints, generate a first input for a generative machine learning model, the first input comprising a first prompt based on the first request, generate, by the generative machine learning model, a first query result for the first input, the first query result comprising a narrative section and a structured section extracted from one or more portions of the data, generate, by the generative machine learning model, at least one suggested follow-on query selectable as a further request, format the first query result and the suggested follow-on query into a first brief summary, and provide the first brief summary to a user interface at the client system.

In embodiments, retrieving the data includes accessing an electronic health record (EHR) to retrieve records including at least one of medical history, records from prior medical visits, laboratory results, diagnostic results, past abnormal observation, previously noted chief complaints, known allergies, medication history, immunization records, insurance information, social history, family history, previous recommendations, past and future appointments, and messages sent through the EHR.

In certain embodiments, accessing the EHR includes limiting a range of the records to items created within a predetermined time period prior to receiving the request. In embodiments, the computer system is further configured to update the data relevant to the first request based on updated information received from the patient at a visit to generate updated data, the updated information including at least one of vitals, one or more chief complaints for the visit, and intake notes at the visit. In embodiments, the computer system is further configured to receive a second request from the client device based on the first brief summary, retrieve data relevant to the second request, the data relevant to the second request including the updated data, generate a second input for the generative machine learning model, the second input comprising a second prompt and one or more portions of the data relevant to the second request, generate, by the generative machine learning model, a second query result from the second input based on the retrieved data relevant to the second request, format the second query result into a second brief summary, and provide the second brief summary to the user interface at the client system.

In certain embodiments, the narrative section includes a first selection from the first query result arranged into natural language text, and the structured section includes a second selection from the first query result used to populate a plurality of preset windows within the first brief summary. In embodiments, at least one of the narrative section and the structured section includes one of a Uniform Resource Locator (URL) and a hovering window to enable display of additional information regarding a specific portion of the first brief summary.

In embodiments, a non-transitory computer-readable media storing computer-executable instructions that, when executed by one or more processors of a computer system, configure the computer system to at least receive a first request from a client system to provide a summary for a patient of a healthcare provider, the summary information comprising patient history information describing at least one of a medical condition and a medical history of the patient, determine at least one of a category of visit and a plurality of medical complaints for the patient, the category including acute, wellness, follow-up, and generic, obtain information regarding a chief complaint for the visit, retrieve data relevant to the first request from a plurality of sources based on the at least one of the category of visit and the plurality of medical complaints, generate a first input for a generative machine learning model, the first input comprising a first prompt based on the first request, generate, by the generative machine learning model, a first query result from the first input, the first query result including a narrative section and a structured section extracted from one or more portions of the data, generate, by the generative machine learning model, at least one suggested follow-on query selectable as a further request, format the first query result and the suggested follow-on query into a first brief summary, and provide the first brief summary to a user interface at the client system.

In certain embodiments, retrieving the data includes accessing an electronic health record (EHR) to retrieve records including at least one of medical history, records from prior medical visits, laboratory results, diagnostic results, past abnormal observation, previously noted chief complaints, known allergies, medication history, immunization records, insurance information, social history, family history, previous recommendations, past and future appointments, and messages sent through the EHR. In embodiments, accessing the EHR includes limiting a range of the records to items created within a predetermined time period prior to receiving the request. In embodiments, the computer system is further configured to update the data relevant to the request based on updated information received from the patient at a visit, the updated information including at least one of vitals, one or more chief complaints for the visit, and intake notes at the visit. In embodiments, the computer system is further configured to receive a second request from the client device based on the first brief summary, retrieve data relevant to the second request, the data relevant to the second request including the updated data, generate a second input for the generative machine learning model, the second input comprising a second prompt and one or more portions of the data relevant to the second request, generate, by the generative machine learning model, a second query result from the second input based on the retrieved data relevant to the second request, format the second query result into a second brief summary, and providing the second brief summary to the user interface at the client system.

In embodiments, the narrative summary includes a first selection from the first query result arranged into natural language text, and the structured section includes a second selection from the first query result used to populate a plurality of preset windows within the first output.

### BRIEF DESCRIPTION OF THE DRAWINGS

The present disclosure is described in conjunction with the appended figures, where like components are indicated with like reference numbers.

FIG. 1 is a block diagram illustrating an example computing environment incorporating agent-driven services, according to certain environments.

FIG. 2 is a block diagram illustrating an example of the function of portions of the cloud service provider platform of FIG. 1, in accordance with embodiments.

FIG. 3 is an example response sent to the client device, in accordance with embodiments.

FIG. 4 is a flowchart illustrating an example process for generating a response in accordance with a user-provided query, in accordance with embodiments.

FIG. 5 is a flowchart illustrating an alternative process for generating a response in accordance with a user-provided query, in accordance with embodiments.

FIG. 6 is a flowchart illustrating a portion of the processes shown in FIGS. 4 and 5, in accordance with embodiments.

FIG. 7 is a block diagram illustrating an example prompt block library, in accordance with embodiments.

FIG. 8 is a block diagram illustrating an example process for filtering semantic objects and generating a response in accordance with a user-provided query, according to embodiments.

FIG. 9 is a flowchart illustrating an example process for using received data in prioritizing information, in accordance with embodiments.

FIG. 10 is a flowchart illustrating another example process generating a response in accordance with received data, according to embodiments.

FIG. 11 is a block diagram illustrating an example computing environment incorporating an agent-driven digital assistant system, in accordance with embodiments.

FIG. 12 is a block diagram illustrating one pattern for implementing a cloud infrastructure as a service system, according to at least one embodiment.

FIG. 13 is a block diagram illustrating another pattern for implementing a cloud infrastructure as a service system, according to at least one embodiment.

FIG. 14 is a block diagram illustrating another pattern for implementing a cloud infrastructure as a service system, according to at least one embodiment.

FIG. 15 is a block diagram illustrating another pattern for implementing a cloud infrastructure as a service system, according to at least one embodiment.

FIG. 16 is a block diagram illustrating an example computer system, according to at least one embodiment.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### Introduction

While a healthcare provider may need to locate and review a variety of information regarding a patient prior to an encounter, it can be difficult to locate, review, and discern the relevant information, even with the proliferation of electronically-accessible patient EHR systems. As a technical challenge, extracting information from a variety of medical record sources with different coding schemes may be difficult for a computing system. For example, many EHR systems use proprietary coding systems for storing patient information such that, while the stored medical information may be standardized within a specific EHR, many different coding schemes are used amongst different EHR systems.

In traditional approaches of computing a summary report in an automated fashion, the generation process is performed in an “end-to-end” fashion, i.e., by retrieving all of the required data from a semantic index (e.g., EHR) and performing a “one-shot” call to a resource such as a large language model. Such an approach is computationally intensive and time consuming.

It is recognized herein that techniques for efficiently generating and updating succinct summaries of patient-specific information to be used during a patient encounter would be desirable.

A useful approach to extracting, filtering, and processing data from disparate sources and generating a summary is the use of digital assistant services using generative artificial intelligence (AI). An example of such an approach is discussed in US Pat. application Ser. No. 18/624,472 to Vishnoi et al., which is incorporated by reference in its entirety herein by reference.

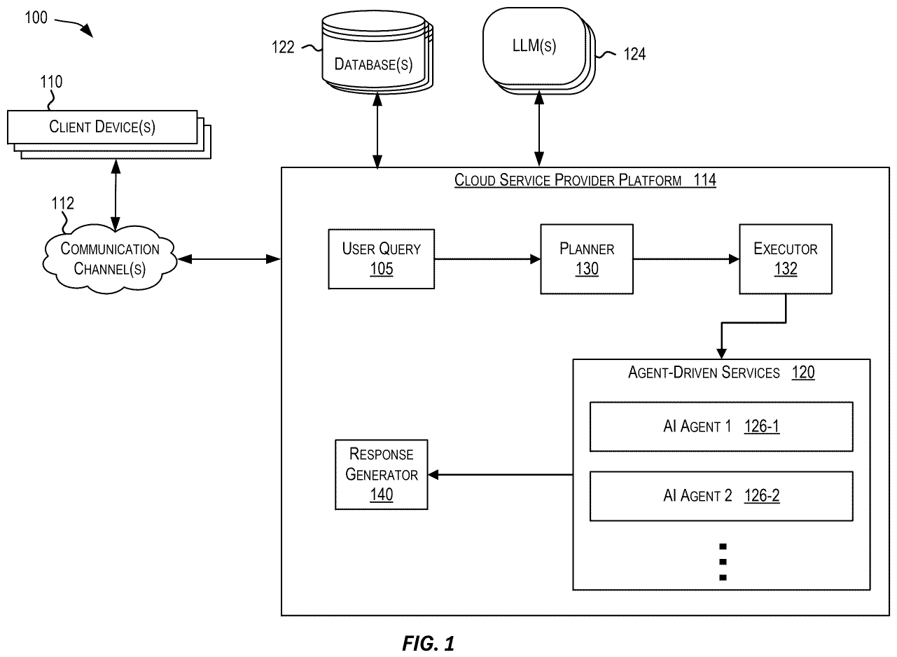

A simplified example of an agentic AI approach to receiving user queries from client devices, extracting data from multiple databases, processing the user queries by managing multiple agent-driven services, then producing a result is illustrated in FIG. 1. FIG. 1 is a block diagram illustrating an example computing environment incorporating agent-driven services, according to certain environments. In examples, the agent-driven services may include one or more artificial intelligence resources acting as “agents,” each performing a defined set of tasks. For instance, one of the agents may implement the summary of patient-specific information, as described herein. In an embodiment, computing environment 100 includes one or more client devices 110 (hereinafter “client devices 110”), one or more communication channels 112 (hereinafter “communication channels 112”), a cloud services provider platform 114 (hereinafter “platform 114”) including agent-driven services 120 and connected with one or more databases 122 (hereinafter “databases 122”) and one or more large language models 124 (hereinafter “LLMs 124”).

While the present disclosure mentions the use of LLMs as an example mechanism for analyzing data and generating patient information summaries, it is noted that other artificial intelligence techniques, including generative artificial intelligence techniques, may be used including, and not limited to, Small Language Models (SLMs), Multi-modal Models, reasoning models and chain-of-thought architectures, transformer-based models, generative machine learning models, and the like.

As shown in FIG. 1, agent-driven services 120 may include, for example, one or more artificial intelligence agents (hereinafter “AI agent 126”). In an embodiment, agent driven services 120 includes a plurality of AI agents, shown as AI Agent 1 (126-1), AI Agent 2 (126-2), and so on, as indicated by ellipsis. Each AI agent may be configured to specialize in a particular task, such as the modular summary generation disclosed herein. In examples, an AI Agent may call one or more of LLMs 124 to generate an execution plan (e.g., instructions), then execute the execution plan. In embodiments, an agent may itself include a model, such as an LLM, small language model (SLM), medium language model (MLM), a machine learning model, or others.

Cloud services provider platform 114 receives user query 105 from one of client devices 110 via communication channels 112, and user query 105 is passed to a planner 130. In embodiments, planner 130 determines the appropriate course of action (e.g., selection of the appropriate AI agent with agent-driven services 120, timing and/or prioritization of tasks to be performed in response to the user query, etc.), then the action so determined is sent to executor 132.

In embodiments, at executor 132, a new execution plan may be generated or an existing plan selected out of a library of execution plans (not shown). An execution plan may include, for example, information regarding the course of action, timing, prioritization, etc. The execution plan is then performed by one or more of the AI agents within agent-driven services 120. The one or more of the AI agents performs the appropriate tasks, based on the information accessible at databases 122 and LLMs 124, to send the resulting output to a response generator 140. Response generator 140 generates then transmits a response to the client device that originated the user query 105.

### Summary Generation System

In contrast with existing AI systems for generating summaries of specific documents or sets of data, the embodiments described herein provide innovative routing models for extracting and filtering data from multiple sources within EHR systems and enabling efficient generation and updating of semantic objects for consumption by medical professionals in clinical settings.

It is noted that, the term “healthcare provider” generally refers to healthcare practitioners and professionals including, and not limited to: physicians (e.g., general practitioners, specialists, surgeons, etc.); nurse professionals (e.g., nurse practitioners, physician assistants, nursing staff, registered nurses, licensed practical nurses, etc.); and other professionals (e.g., pharmacists, therapists, technicians, technologists, pathologists, dietitians, nutritionists, emergency medical technicians, psychiatrists, psychologists, counselors, dentists, orthodontists, hygienists, etc.).

The resulting semantic object may be in the form of a summary report, presenting the most relevant information including structured and unstructured data necessary for an anticipated patient encounter. The extraction, transformation, and filtering of the relevant data allows on-the-fly updating of the summary report essentially in real time, as new information is discovered during the patient encounter. The summary report also integrates links to additional information within the EHR or elsewhere to provide further detail regarding specific documents, conditions, terminology, and other portions of the extracted data.

The developed approach described herein addresses these challenges and others by providing techniques for operating an agent-driven digital assistant system in efficiently generating summaries of patient information while reducing the computing resources required to and update the summaries of necessary information in a clinical environment. In particular, the techniques disclosed below provide efficient systems and methods for extracting and presenting relevant information related to a patient encounter in a useful format, while enabling quick regeneration of the presentation as additional or different information is discovered during the patient encounter.

Further, the presently disclosed approach processes each semantic object as few times as possible, preferably only once. Relevant collections of data, as extracted from the EHR and other database resources based on knowledge regarding an anticipated patient encounter, may be cached prior to the patient encounter, thus enabling computationally efficient generation and regeneration of patient summaries, tailored to the information required for a particular patient encounter. In this way, the initial generation and updated regeneration of patient summaries are efficiently producible with much reduced latency. Further, the extracted facts may be additionally processed for value-add activities such as trend detection (such as application of “sliding window” algorithms), incremental updating of selected, prioritized data (such as most recently diagnosed conditions and medications), and recommended actions (e.g., providing suggestions for recommended next steps), thus enabling efficient, effective, and useful presentation of necessary information in a variety of patient encounter settings.

Embodiments of the present techniques may be implemented as a part of a cloud service provider platform including a plurality of automated agent-driven services, such as illustrated in FIG. 1.

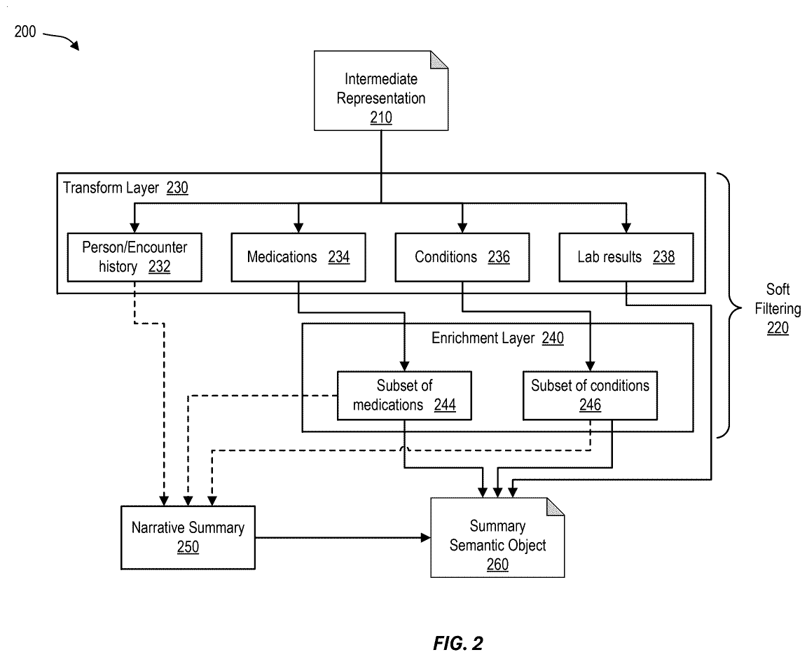

FIG. 2 is a block diagram illustrating an example of the function of portions of the cloud service provider platform of FIG. 1, in accordance with embodiments. The initial patient summary may be suitable, for example, for use as a pre-visit summary report to be quickly reviewed by a healthcare provider prior to a scheduled patient encounter or in preparation for an acute medical visit.

As shown in FIG. 2, a system 200 begins with an intermediate representation (IR) 210 of the relevant data, with the IR having a structured, computer-interpretable encoding format as used in subsequent natural language (NL) processing. The intermediate representation may be produced, for example, by extracting data (e.g., known facts, notes, etc.) specific to the patient from one or more databases, such as an Electronic Health Record system, medical databases, other business records accessible to the medical practice, public resources, and others. The IR may also take into consideration a priori knowledge regarding the recorded chief complaint for an anticipated patient encounter, contextual information. The use of semantic objects from an EHR system as the source of IR data may be particularly advantages as the semantic objects align closely with Fast Healthcare Interoperability Resources (FHIR) standards, thus providing ready adaptability to multiple EHR systems and other data resources following the FHIR standard.

In examples, each note in the databases will be processed once, with the extracted facts being stored in IR form for subsequent retrieval in generating the summaries. In this way, the NL content of each note is not required to be stored, thus reducing computational cost in subsequent processing as the extracted facts in IR form may be directly used as the basis of the summarization prompts.

A variety of information may be extracted as IR 210 such as, and not limited to:
Name
Age
Gender
Reason for visit (RFV)
Chief complaint (CC)
Last Visit Date and Type
Related Visit Notes Since Previous Visit
New Related Problems Since Previous Visit
New Related Medications Since Previous Visit
Current and New Allergies Since Previous Visit
New Related Diagnostic Study Data and Lab Results Since Previous Visit
New Related Procedures
Related Social History
Related Family History
Current Vitals (if already taken at this visit)
Current Nurse Notes (if already started this visit)
Recommendations
Future related appointments
Current Related Messages from Message Center

Continuing to refer to FIG. 2, the IR 210 is directed through a sequence of soft filtering 220, including transformation of IR 210 using generic prompts and queries. In an example, soft filtering 220 includes processing IR 210 through a transform layer 230, in which aspects of IR 210 are filtered for commonly relevant facts. For instance, transform layer 230 includes a personal history/encounter history module 232 configured to extract information that may be relevant for the visit. Similarly, IR 210 may be processed through other filters such as medications 234 (e.g., for prescription history), conditions 236 (i.e., history of known conditions and diagnoses), and lab results 238 (e.g., laboratory results for related conditions, known lab results for a given time period (e.g., in the past six months)). As personal and encounter history are generally recorded in EHR systems as unstructured data in note format, such data may be particularly processed in a manner compatible with unstructured data, such as using character and word recognition, chunking, sectionizing, and other known techniques. Other information, such as medications, conditions, and lab results, are generally stored in a more structured manner, including links to external resources (e.g., a drug manufacturer's webpage, federal Food and Drug Administration registers, etc.) and numerical data in predetermined formats.

Portions of the filtered information out of transform layer 230 may be further passed through an enrichment layer 240 to filter for more specific information. For instance, the medication-related information from transform layer 230 may be further gleaned for relevant data given the output from conditions 236, to extract a subset of medications (block 244) known to have a therapeutic or adverse effect on known conditions. Similarly, a subset of conditions (block 246) may be extracted to highlight conditions known to be affiliated with the extracted medications and other a priori information extracted in transform layer 230. It is noted that, while enrichment layer 240 may involve a large language model, other techniques for further processing information may be used, such as a knowledge graph (e.g., Semantic Knowledge Graph (SKG)), trained machine learning models, keyword filters, and others.

The extracted portions of data from transform layer 230 and enrichment layer 240 may be used in crafting a narrative summary 250. For example, extracted IR data from soft filter 220 (with an example flow of data indicated by dashed arrows) may be compiled through natural language processing methods to generate narrative summary 250 based on the extracted information. In examples, narrative summary provides a pithy yet informative summary of patient information, particularly highlighting patient and encounter history as well as subset of medications and conditions relevant to the patient encounter. Especially for medical professionals who are used to having patient information presented to them in narrative form, narrative summary 250 provides an effective way to convey the necessary information, accurately weaving in the relevant patient information in natural language.

Narrative summary 250 may be provided as a standalone report and/or combined with other facts extracted from IR 210, depending on the summary type and use case, to generate a summary semantic object 260. For instance, in addition to a narrative summary section, structured data may also be used to populate a structured portion of summary semantic object 260. The structured data may include, for example, facts and numerical information as related to known medications, lab results, and other structured information that may be directly inserted into a form, added as a universal resource locator (URL) link, etc.

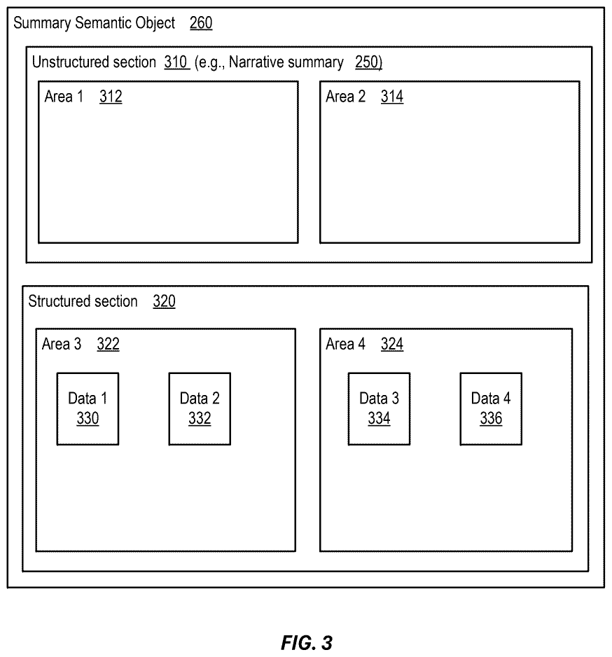

An example presentation format of summary semantic object 260 is shown in FIG. 3. As shown in FIG. 3, which shows an example response sent to the client device, in accordance with embodiments, summary semantic object 260 includes an unstructured section 310 (including, as an example, narrative summary 250 of FIG. 2). Unstructured section 310 may include one or more areas (shown as area 1 (312) and area 2 (314)), into which narrative related to specific topics may be inserted. For instance, area 1 (312) may be used to present a general summary of a patient's current chief complaints, while area 2 (314) may be reserved for a summary of patient's family, conditions, and medication history.

The information presented in unstructured section 310 may be an extract from narrative summary 250 or independently populated using extracted IR 210. Unstructured section 310 may include a generated summary of the relevant information in natural language format, bullet points of key information, extracts from notes from previous patient encounters, electronic scans of historical notes, and other long-form information.

Summary semantic object 260 may additionally include a structured section 320. In the illustrative example, structured section 320 may include one or more areas (shown as area 3 (322) and area 4 (324)), in which structured data such as lab test results and medication lists may be presented. Area 3 (322) and area 4 (324) may include structured data extracted from IR 210 used to populate an predetermined template. For example, area 3 (322) may include data 1 (330) presented as a list of past patient conditions next to data 2 (332), including a list of medications relevant to the past patient conditions, or a list of previous Assessment & Plans (A&Ps) as frequently referenced in patient charts. Similarly, area 4 (324) may include data 3 (334), with a graph visually showing the evaluated numbers from a series of blood test results, along with data 4 (336) with a list of links containing URLs linking to external or internal repository of information related to explanation of the blood test results. Such visual representation of structured data may assist the healthcare provider in identifying trends as related to the rest of the patient medical history. Optionally, the information presented in the structured section may have been further processed, for example, to emphasize the most recently entered information, such as the list of active prescriptions or lab test results over the past six months.

In this way, summary semantic object 260 presents a snapshot of the patient's past history in a compact format, suitable for display on a small screen such as a tablet, as well as links to additional information of so desired. In embodiments, one or more portions of summary semantic object 260 may be reserved to allow the healthcare provider to enter additional notes.

In certain embodiments, summary semantic object 260 may include a search field or a user interface “button” to allow the healthcare provider to regenerate the summary semantic object based on any newly added information and the previously extracted IR 210. If necessary, additional information may be pulled from one or more databases to be added to IR 210. In certain embodiments, the healthcare provider may be presented with an option to save the current summary report to a file folder in memory, prior to generating an updated summary report. For instance, as a particular patient may present with multiple medical conditions, additional and/or updated summary reports may be necessary for a complete assessment of the patient history.

As a further example, summary semantic object 260 may incorporate into summary semantic object 260 one or more selectable follow-on queries presented to the user. For instance, if subset of medications 244 and subset of conditions 246 as processed at enrichment layer 240 of FIG. 2 include potentially conflicting information, such as the patient having been prescribed medications for a first condition that may have an adverse effect on a second condition with which the patient has been separately diagnosed, summary semantic object 260 may include a clickable link selectable by the user to obtain more information (e.g., via a pop-up window) on the medication or conditions in question and/or to re-generate the summary semantic object taking into consideration the potential implications of the combination of the medication and the conditions in question. Similarly, summary semantic object 260 may include suggested follow-on queries such as, “An alternative formulation of medication ______ is available. Would you like more information? ” or “New lab results for ______ analysis was received ______ minutes ago. Generate updated summary? ” In other cases, enrichment layer 240 may notice correlations between the extracted IR and provide an alert as part of summary semantic object 260, such as “Patient has multiple prescriptions to medication ______. Click here [include URL link] for list of existing prescriptions. ” Such a “suggested next” may be provided by, for example, by enrichment layer 240 or one of LLM(s) 124 of FIG. 1.

In embodiments, one of the areas of summary semantic object 260 may be reserved for presenting recommendations for next steps of action for the patient. Such recommendations may be generated, for example, based on prior knowledge of the patient history, industry standard courses of action, latest guidance from regulatory and industry standard organizations, and others. As additional examples, certain portions of the unstructured section may be enhanced with structured information, such as URL links to access external documents (e.g., images of handwritten notes).

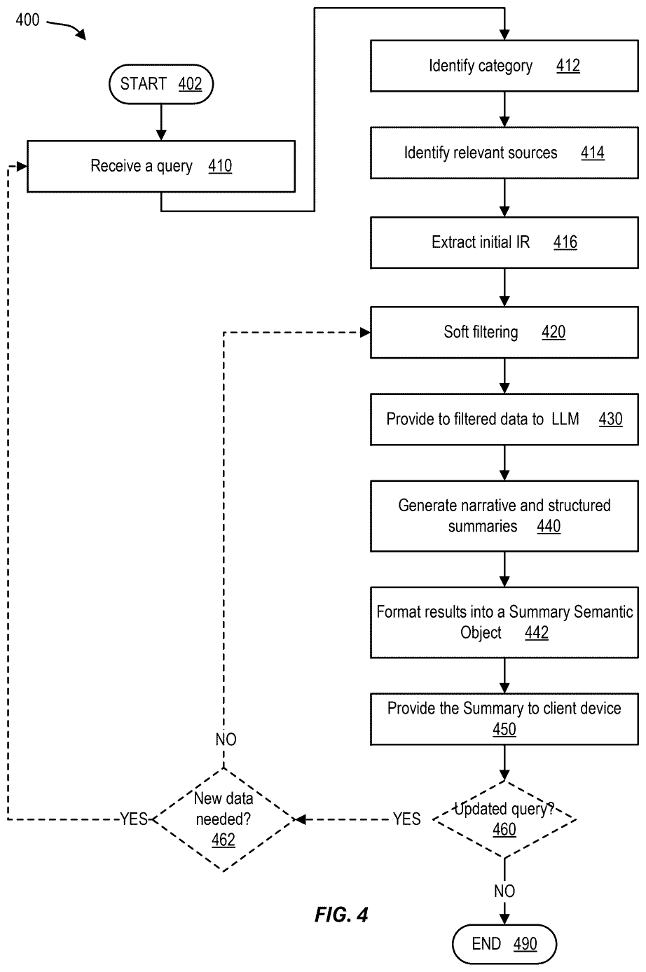

An example process suitable for use in generating patient summaries as described above is illustrated in FIG. 4, showing a flowchart illustrating an example process for generating a response in accordance with a user-provided query, in accordance with embodiments. As shown in FIG. 4, a process 400 may be performed in a computing environment (e.g., by cloud service provider platform 114 of FIG. 1). Process 400 is initiated in a start step 402, then proceeds to receive a query in 410. The query may have been received from a client device (e.g., client device 110).

In the example process 400, the query is used to identify a category of the patient encounter in block 412. The category may be selected from preset categories such as an acute/urgent care visit, an annual/wellness visit, a follow-up visit as related to a previous encounter, and a generic/uncategorized visit. In block 414, the process proceeds to identifying the relevant sources of data from which the relevant data should be pulled. For example, if a pediatric patient is scheduled for an annual wellness visit, then one of the relevant data sources may be the EHR containing information regarding previously administered immunization records as well as federal and industry guidance on currently recommended immunization schedules, along with age, known conditions, and family history for the specific patient. The initial intermediate representations are extracted from the identified relevant sources in block 416.

The IRs as extracted are then soft filtered in block 420, such as by using an environment as illustrated in FIG. 2. For example, soft filtering 420 may include applying one or more topic-specific soft filters and enrichment (e.g., based on history, meds, conditions, labs, and, optionally, semantic knowledge graphs or predefined rules) to the IR before model invocation in the following steps.

At least a portion of the filtered data may be provided to a generative resource, such as a trained LLM (e.g., LLM(s) 124 of FIG. 1), in block 430, which in turn generates narrative and structured summaries in block 440. The results of block 440 may then be formatted into a Summary Semantic Object (e.g., as shown in FIG. 3) in block 442, and the summary report is provided to the client device or the originator of the query in block 450.

Multiple approaches are contemplated for the generation of the narrative and structured summaries and are considered to be a part of the present disclosure. For example, zero-shot prompting, one-shot prompting, chain-of-thought prompting may be considered for all or different aspects of the summary semantic object generation. Additional considerations to enhance the summary narrative may include generating the sections of the narrative in parallel, using a separate yet smaller LLM call for each section, thus reducing latency.

Optionally, a decision may be made in a determination 460 whether an updated or new query related to the specific patient and/or patient encounter has been received, such as via the client device user interface, based on a search performed by the healthcare provider reading the summary report, clickthroughs to URLs provided as part of the summary report, and other information. If an updated query has been received, then a determination 462 is made whether new or additional data is required to respond to the updated query. If determination 462 results in YES, new data is needed (e.g., if a new condition or medication has been identified), then process 400 returns to block 410 to process the updated query anew. If determination 462 results in NO, new data is not needed and the new query may be addressed using the extracted initial IR, the process 400 returns to soft filtering block 420, thus enabling savings in computational time and resources. If no updated query has been received, then process 400 is terminated at end step 490.

Various modifications to process 400 may be contemplated and are considered to be a par of the present disclosure. For example, rather than exclusively extracting the relevant IRs in block 416, process 400 may include extraction of any potentially relevant information (not necessarily IR) from the identified relevant sources, then format the extracted data into IR format, such as Fast Healthcare Interoperability Resources (FHIR)-aligned intermediate representation. Additionally, the extracted IR may be persisted at the computer system for reuse across related requests and/or for responding to future requests for updated summary information, thus further reducing latency in regenerating the summaries. In generating block 440, the LLM may be provided with pre-crafted prompt blocks that have been selected based on, for example, a predicted or identified visit category and/or chief complaint. Further, the narrative and structured sections may be generated in parallel (e.g., as shown in FIG. 2) using separate LLMs. Further, in examples, the IR and/or selected processing modules (e.g., used for soft filtering 420) may be cached for future use such as to address second and further updated requests, with the cache being keyed to be specific to a specific patient and/or encounter with defined invalidation triggers (e.g., time, closing of the encounter records, patient “checkout” from the healthcare provider practice).

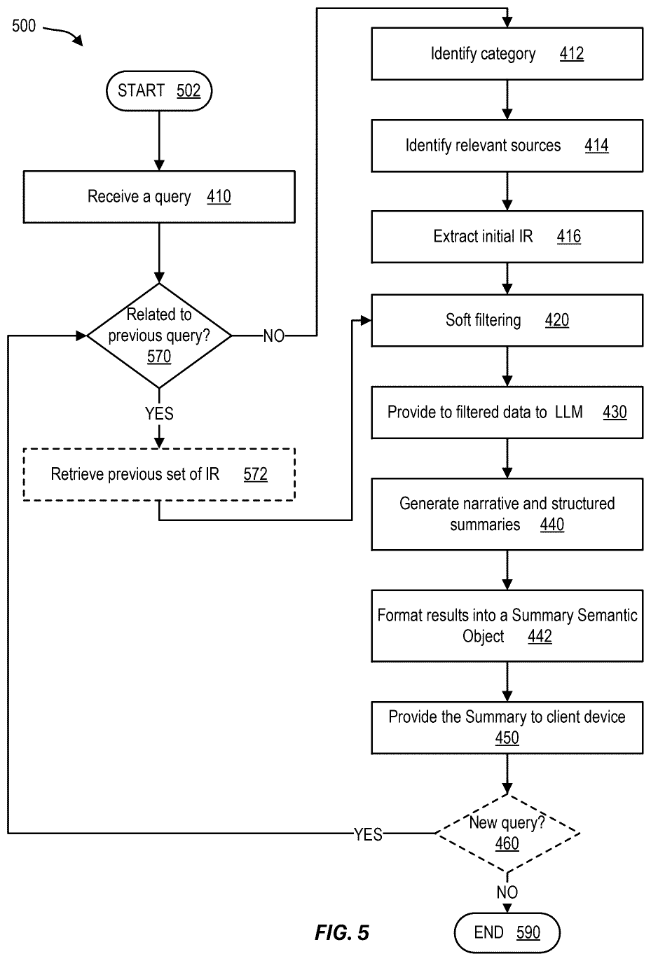

FIG. 5 illustrates an alternative process in which every query is evaluated whether it is related a previously presented query. As shown in FIG. 5, which is a flowchart illustrating an alternative process for generating a response in accordance with a user-provided query, in accordance with embodiments, a process 500 shares several of the processing blocks as shown in FIG. 4. However, upon initiating process 500 at a start step 502 and a query is received in block 410, a determination 570 is made whether the received query is related to a previously presented query. If determination 570 results in NO, that the received query is unrelated to previously submitted queries, then process 500 proceeds to block 412 and follows the processing steps as shown in FIG. 4.

If determination 570 yields YES, the presently received query is related to a previous query, then process 500 optionally proceeds to retrieve a previously extracted set of IR (e.g., from cached records at the client device or within the cloud services platform) in block 572 then to soft filtering block 420. Alternatively, for example if the previously extracted IR is still active at the client services platform, then process 500 may immediately proceed to perform soft filtering anew at block 420.

The techniques described in FIGS. 4 and 5 allows efficient production of multiple summaries for a given patient, while processing each semantic object as few times as possible. For example, information for a given patient may be cached and shared as at least a portion of the extracted IR in addressing a new or updated query. The shared information may include, for example, conditions or medications for the patient, lab tests or vitals relevant to different conditions, family history, and previous summarized A&P from previous patient encounters. In embodiments, when a summary generation is requested, then the pre-computed modules (i.e., extracted and/or filtered IRs) may be loaded to maximize reuse of semantic objects, then the patient summaries may be generated using minimal computational resources (e.g., LLM tokens).

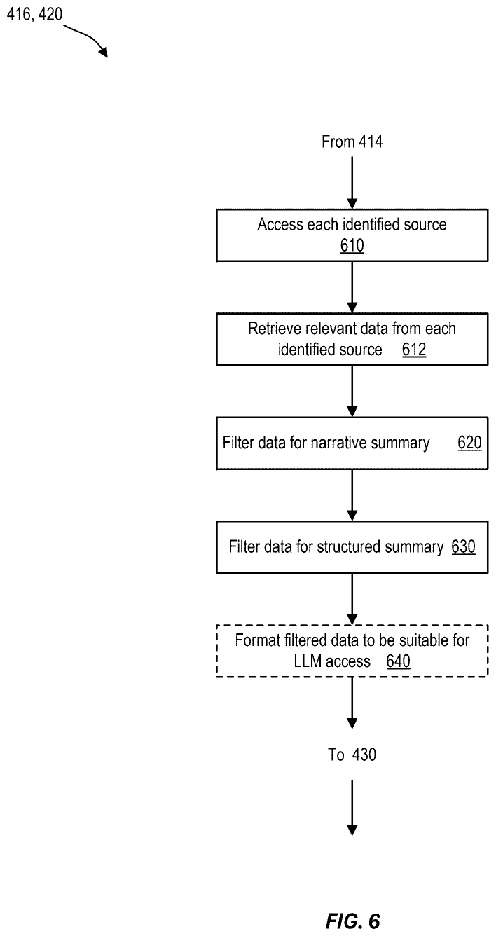

FIG. 6 shows additional details of the processing performed at blocks 416 and 420 of FIGS. 4 and 5, in an embodiment. As shown in FIG. 6, which shows a flowchart illustrating a portion of the processes shown in FIGS. 4 and 5, in accordance with embodiments, after relevant sources are identified in block 414, each identified source is accessed in block 610, and relevant data retrieved in block 612, such as to collect IR 210 of FIG. 2. Then the retrieved relevant data are filtered for use in generating the narrative summary in block 620 as well as for use in the structured summary section in block 630, such as illustrated in FIGS. 2 and 3. The filtered data may optionally be formatted for LLM access in block 640. The formatting is considered optional as the extracted IR may already be in a format suitable for post processing by an LLM or other mechanisms, as discussed above. The process proceeds to block 430 of FIGS. 4 and 5 to continue with the patient summary generation process.

In embodiments, a prompt block library may be provided at the soft filtering stage (e.g., soft filtering 220 of FIG. 2) in order to further reduce computational cost. Pre-crafted prompts by category or filtering topic from the prompt block library may be selected according to predefined query categories such that the LLMs may be initially trained using the pre-crafted prompts in generating patient summaries that fit the needs of specific use case scenarios.

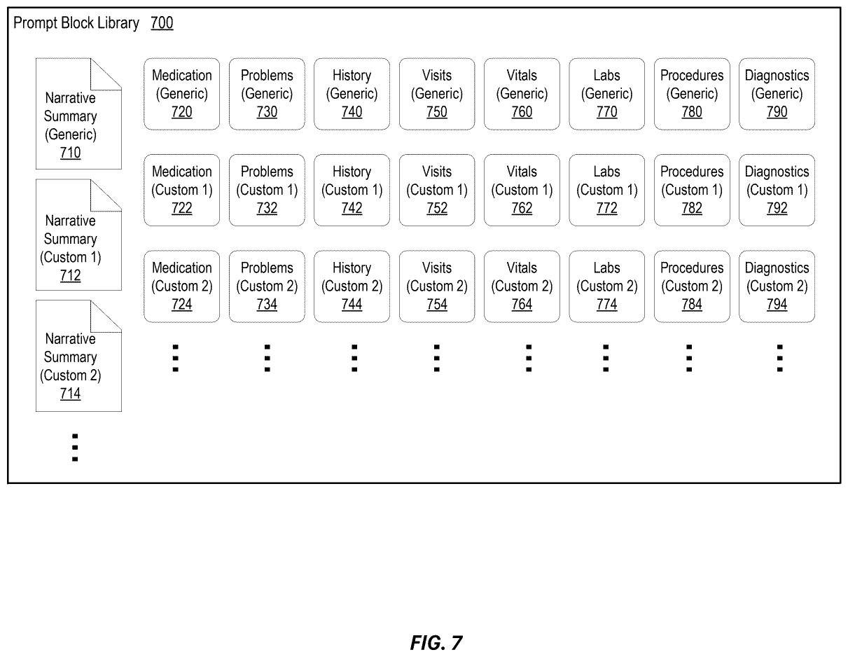

FIG. 7 is a block diagram illustrating an example prompt block library, in accordance with embodiments. As shown in FIG. 7, a prompt block library 700 may include a plurality of pre-crafted, prompt blocks for determining relevant information from the extracted IRs. For instance, the prompt blocks may be used in the soft filtering stages of system 200 of FIG. 2 to filter IR 210 for data relevant to specific queries.

Prompt block library 700 includes prompt blocks related to a variety of topics, such as medication, problems (e.g., chief complaint and/or reason for visit), history (e.g., patient history, family history, allergies, etc.), previous visits, vitals, laboratory results, previously performed procedures, and previous diagnostics. Each prompt block may be crafted for generic situations (e.g., healthy adult male in his 50s with an appointment for an annual wellness checkup) and/or customized for specific queries according to known information for a particular patient. Additionally, prompts for generating narrative summaries from unstructured data for a variety of situations may also be included in prompt block 700.

In the illustrated example, prompt block library 700 includes pre-crafted prompts related to handling structured and unstructured IR data for narrative summary generation (generic (710), custom 1 (712), custom 2 (714), and so on as indicated by ellipsis), medication history (generic (720), custom 1 (722), custom 2 (724), and so on), known problems (generic (730), custom 1 (732), custom 2 (734), and so on), patient and family history (generic (740), custom 1 (742), custom 2 (744), and so on), previous visits (generic (750), custom 1 (752), custom 2 (754), and so on), vital signs as recorded at the patient encounter (generic (760), custom 1 (762), custom 2 (764), and so on), lab results (generic (770), custom 1 (772), custom 2 (774), and so on), known previous procedures (generic (780), custom 1 (782), custom 2 (784), and so on), and known diagnostics (generic (790), custom 1 (792), custom 2 (794), and so on). Prompts related to other relevant topics may also be pre-crafted and recorded in prompt block library 700.

Rather than generating prompts from each query, a selected set of pre-crafted prompts from prompt block library 700 may be used in generating patient summaries for specific use case as gleaned from the received query based on known patient information. The selected set of prompts may then be used to assess the extracted IR to further narrow the range of data required in the analysis.

For example, for a given query related to a healthy patient arriving for a wellness visit, the summary generation system may select a combination of pre-crafted prompts, such as Narrative Summary (Generic) 710, Medication (Generic) 720, Problems (Generic) 730, History (Generic) 740, Visits (Custom 1) 752, Vitals (Generic) 760, Labs (Generic) 770, Procedures (Generic) 780, and Diagnostics (Generic) 790. The combination of prompts selected for a different patient with a history of known conditions and treatments would be quite different. However, by having the pre-crafted prompts and only generating new prompts when a particular situation lies outside of the anticipated scenarios, the summary generation system of the present disclosure can save processing time and computational cost in the initial and subsequent updated summaries. Additional categories of pre-crafted responses may also be contemplated and are considered a part of the present disclosure.

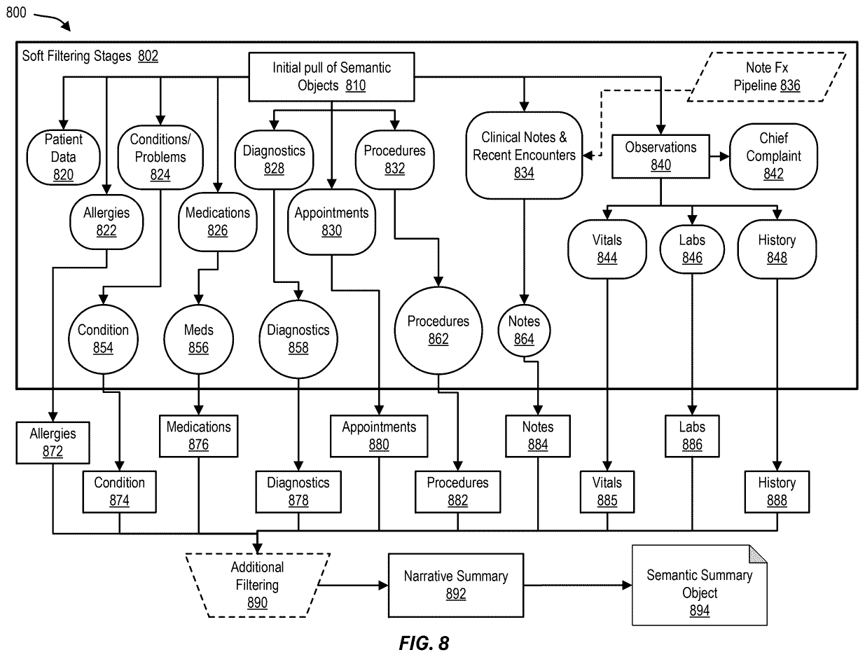

FIG. illustrates an alternative patient summary generation system, in accordance with embodiments. The example illustrated in FIG. 8 may be particularly applicable for a patient arriving for an acute visit, for example. As shown in FIG. 8, which shows a block diagram illustrating an example process for filtering semantic objects and generating a response in accordance with a user-provided query, a system 800 includes soft filtering states 802, which takes an initial pull of semantic objects (e.g., IR 210 of FIG. 2), then filters the data in accordance with a variety of filters, such as patient data 820, allergies 822, conditions/problems 824, medications 826, diagnostics 828, previous and existing appointments 830, known previous procedures 832, and clinical notes and recent encounters 834. In an example, unstructured data corresponding to the clinical notes and recent encounters may be obtained from a separate note filtering (Fx) pipeline 836 to the unstructured data section of one or more EHR systems, such that note Fx pipeline may perform pre-processing of unstructured data into more readily ingestible formats, thus improving the efficiency of obtaining such data.

Observations 840 by a healthcare provider during a patient encounter may also lead to an identification an appropriate filter for chief complaint 842 as well as vitals 844, lab results 846, and patient and family history 848. Such filtering may be equivalent to operations performed, for example, in the transform layer 230. Further the filtering may be performed using a combination of pre-crafted prompts in the prompt block library, as shown in FIG. 7 to reduce computational load and improve the predictability of the filter outputs, particularly if the transform layer has previously been trained on the prompt blocks in the library.

Additional refining on the results of the filtering may be performed in certain sets of data (i.e., in a manner equivalent to the functions performed in enrichment layer 240 of FIG. 2), such as condition 854, medications 856, diagnostics 858, procedures 862, and even clinical notes 853. The resulting filtered information (e.g., allergies 872, condition 874, medications 876, diagnostics 878, appointments 880, procedures 882, notes 884, vitals 885, labs 886, and history 888 in the illustrated example) may optionally be subject to additional filtering 890, and the results are used to generate a narrative summary 892, which becomes a part of an overall semantic summary object 894 in combination with the structured and unstructured data formatted in a predefined presentation format. For instance, the predefined presentation format may include predefined areas of the semantic summary object 894 into which specific filtered information may be slotted so as to provide a predictable location for a healthcare provider to easily locate frequently used pieces of information. As an example, the patient name and age may be assigned to always appear in the opening sentence of the narrative shown in Area 1 (312), while most recent vitals information is always shown as Data 3 (334) in Area 4 of summary semantic object 260 of FIG. 3.

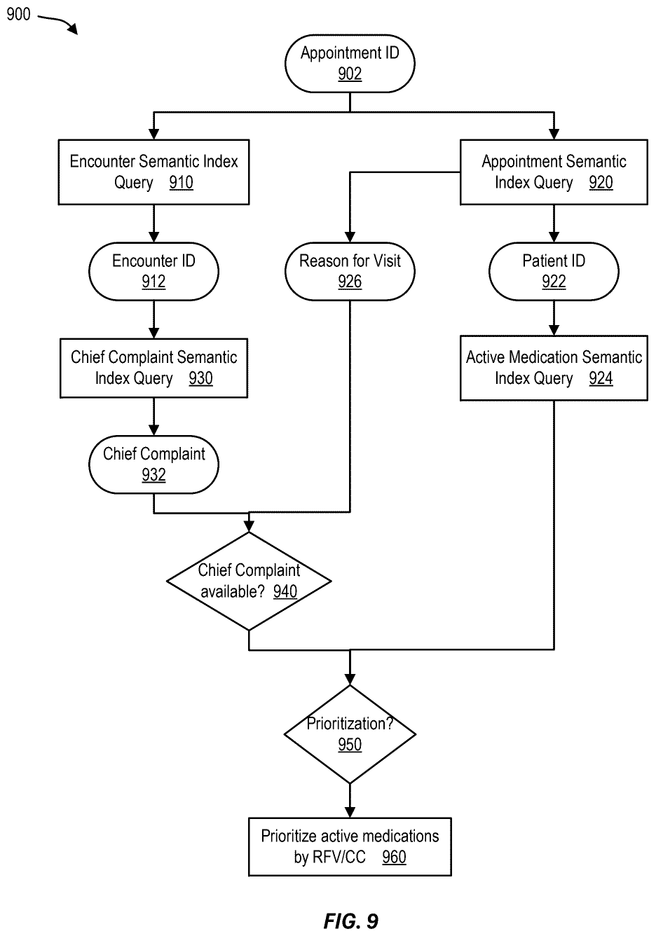

Still another example of a process flow is shown in FIG. 9, in accordance with embodiments. The illustrated example in FIG. 9, showing a flowchart illustrating an example process for using received data in prioritizing information, in accordance with embodiments, may be applicable, for example, in preparing for an encounter with a patient being evaluated for a new medication.

As shown in FIG. 9, process 900 begins with the creation of an appointment ID 902. The appointment ID creation may be performed automatically when a patient schedules an appointment via a patient portal or if scheduled by office staff at a medical practice. Appointment ID information may be used to create an encounter semantic index (SI) query in 910 to create an encounter ID 912. In parallel, an appointment SI query creation in 920 may be used to identify a patient ID 922, which may be used in performing an active medication SI query 924, as well as a stated reason for visit 926 as reported by the patient when making the appointment. The encounter ID information may be used in performing a chief complaint SI query to pull information related to previously reported chief complaints, if any, associated with the specific patient.

The encounter ID, patient ID, and stated reason for visit may be used as the basis for extracting relevant IR information from relevant databases on which the queries 910, 920, 924, and 930 may be made. If a chief complaint has been previously reported, as determined in 940, the CC information may be combined with the active medication SI query result to determine a prioritization of the medication to be considered for the scheduled patient encounter. For example, the summary report generated from a determination of prioritization of the candidate medications in 950 may prioritize the currently active medications as related to the previously documented chief complaint, given the results of the active medication SI query. In other situations, the acuteness of the reason for visit or the specific conditions being treated with the currently active medication may also be considered in the prioritization used in suggesting potential medications and/or A&P as anticipated for the patient encounter. It is noted that the process of FIG. 9 may be repeated as new relevant information may be obtained prior to, during, or following the scheduled appointment.

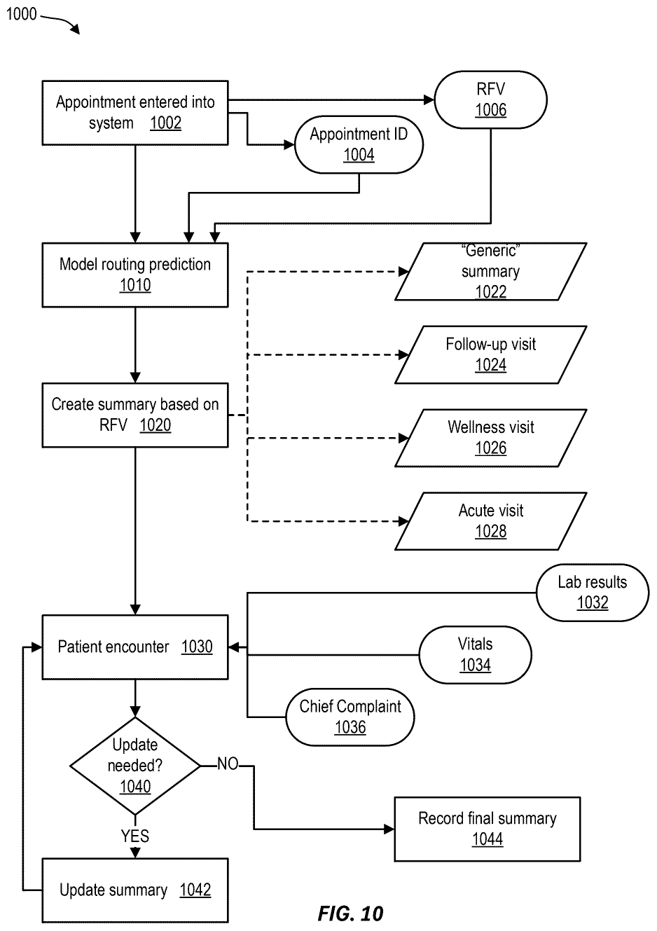

A routing example based on appointment type is shown in FIG. 10, showing a flowchart illustrating another example process generating a response in accordance with received data, according to embodiments. As shown in FIG. 10, a process 1000 begins when an appointment is entered into a cloud service provider platform (e.g., cloud service provider platform 114 of FIG. 1), which leads to the generation of an appointment ID in 1004 and a recordation of a reason for the visit 1006. The appointment ID and RFV information are processed by a model routing prediction block 1010, which generates a prediction of the type of appointment (e.g., acute/urgent care, annual/wellness, follow-up, and generic, as categories discussed above). Based on the prediction, process 1000 creates an initial patient summary in block 1020, such as a “generic” summary 1022, a follow-up visit summary 1024, a wellness visit summary 1026, and an acute visit summary 1028, given the IR and routing prediction based on the appointment ID and RFV information. The summary creation may be based, for example, on the selection of pre-created prompt blocks for the predicted encounter scenarios. The various summaries may be created then selected by a healthcare provider prior to the encounter, or one or a few of the summaries may be created in anticipation of the patient encounter according to the predictions.

Upon conducting the patient encounter in block 1030, the healthcare provider may choose to enter additional information for consideration in the summary generation, such as new or existing lab results 1032, current vitals 1034, and reported chief complaint 1036. A determination 1040 is made whether an updated summary is needed. If necessary, an updated summary is created with the new information in block 1042. If no update to the patient summary is deemed to be required, then the selected final summary may be recorded into the patient records.

It is noted that the extracted IR and filtered information used in the generation of the summary may also be used to suggest next steps, such as modifications to a summary report based on new information obtained during the patient encounter, identification of additional information that may be useful for a given patient encounter, and others. For example, the initial summary may note that, given the patient information available, it may be useful for the healthcare provider to view the A&P notes as recorded at the last appointment with the same patient. Similar suggestions on “suggested next” may be generated also based on the extracted IR, rather than the entire body of information available to the cloud service provider platform.

The developed approach described herein addresses these challenges and others by providing techniques for assisting healthcare providers with necessary and time-consuming and often tedious tasks. Techniques are disclosed herein for improving the efficiency of and reducing the computing resources required to perform various healthcare services in a clinical environment. In certain embodiments, techniques are disclosed for equipping a healthcare provider end user with a clinical software application that can be installed on and utilized from one or both of a mobile computing device and a desktop computing device to facilitate performance of the various tasks typically rendered by a healthcare provider as part of providing healthcare services to patients.

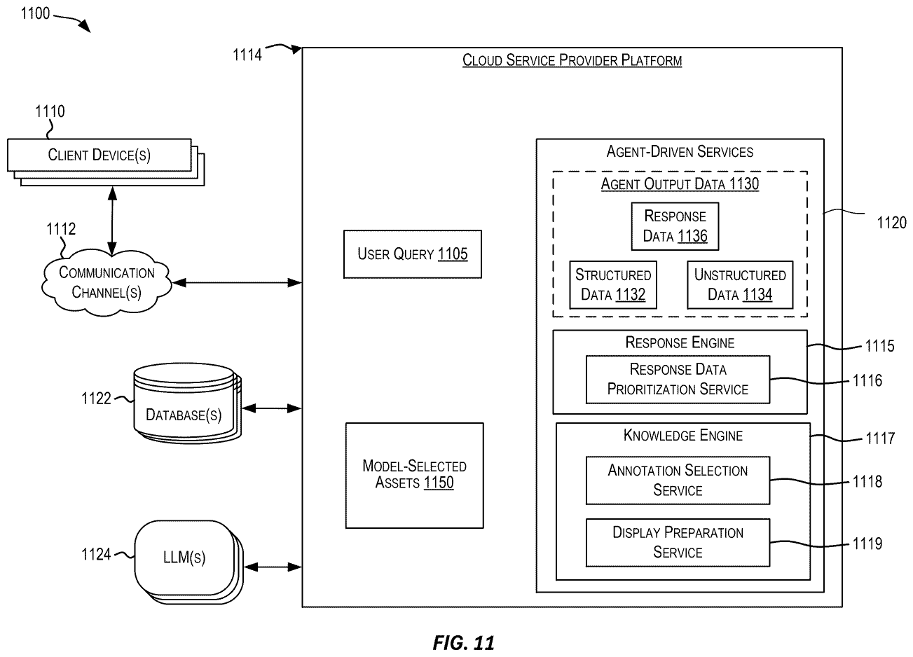

FIG. 11 shows a simplified diagram depicting a computing environment 1100 incorporating an agent-driven digital assistant system configured to generate knowledge-grounded response data according to certain embodiments. As shown in FIG. 11, the computing environment 1100 includes one or more client devices 1110 (hereinafter “client devices 1110”), one or more communication channels 1112 (hereinafter “communication channels 1112”), a cloud service provider platform 1114 (hereinafter “platform 1114”), one or more databases 1122 (hereinafter “databases 1122”), and one or more LLMs 1124 (hereinafter “LLMs 1124”). The platform 1114, which can be included as part of a cloud infrastructure of a cloud service provider (e.g., Oracle Cloud Infrastructure or OCI), can be configured to communicate with, send data and information to, and receive data and information from the client devices 1110 via the communication channels 1112. Additionally, the platform 1114 can be configured to access and/or call the databases 1122 and the LLMs 1124 to obtain and/or receive data and information from the databases 1122 and the LLMs 1124. Data and information received from the client devices 1110, the databases 1122, and the LLMs 1124 can be used by the platform 1114 to execute tasks and perform services such as automatically generating one or more portions of knowledge-grounded response data. While FIG. 11 shows the databases 1122 and the LLMs 1124 as being separate from the platform 1114, this is not intended to be limiting, and one or more of the databases 1122 and/or one or more of the LLMs 1124 can be included as part of the platform 1114 and/or the cloud infrastructure in which the platform 1114 is included. While FIG. 11 describes the computing environment 1100 as including the LLMs 1124, other types of ML models can be included in the computing environment 1100, such as an ML model configured for analyzing audio data and/or generating text based on audio data or an ML model configured to generate an execution plan for a group of multiple agent-driven services (or sub-services) included in the platform 1114.

Each client device included in the client devices 1110 can be any kind of electronic device that is capable of: executing applications; presenting information textually, graphically, and audibly such as via a display and a speaker; collecting information via one or more sensing elements such as image sensors, microphones, tactile sensors, touchscreen displays, and the like; connecting to a communication channel such as the communication channels 1112 or a network such as a wireless network, wired network, a public network, a private network, and the like, to send and receive data and information; and/or storing data and information locally in one or more storage mediums of the electronic device and/or in one or more locations that are remote from the electronic device such as a cloud-based storage system, the platform 1114, and/or the databases 1122. Examples of electronic devices include, and are not limited to, mobile phones, desktop computers, portable computing devices, computers, workstations, laptop computers, tablet computers, and the like.

In some implementations, an application can be installed on, executing on, and/or accessed by a client device included in the client devices 1110. The application and/or a user interface of the application can be utilized and/or interacted with (e.g., by an end user) to access, utilize, and/or interact with one or more services provided by the platform 1114. The client devices 1110 can be configured to receive multiple forms of input such as touch, text, voice, and the like, and the application can be configured to transform that input into one or more messages which can be transmitted or streamed to the platform 1114 using one or more communication channels of the communication channels 1112. Additionally, the client device can be configured to receive messages, data, and information from the platform 1114 using one or more communication channels of the communication channels 1112 and the application can be configured to present and/or render the received messages, data, and information in one or more user interfaces of the application. In some cases, the platform 1114 receives one or more user queries, such as a user query 1105, from the client devices 1110. In some cases, the platform 1114 provides one or more knowledge-grounded responses, such as knowledge-grounded response data 1190, to the client devices 1110.

Each communication channel included in the communication channels 1112 can be any kind of communication channel that is capable of facilitating communication and the transfer of data and/or information between one or more entities such as the client devices 1110, the platform 1114, the databases 1122, and the LLMs 1124 (or other ML models). Examples of communication channels include, and are not limited to, public networks, private networks, the Internet, wireless networks, wired networks, fiber optic networks, local area networks, wide area networks, and the like. The communication channels 1112 can be configured to facilitate data and/or information streaming between and among the one or more entities. In some implementations, data and/or information can be streamed using one or more messages and according to one or more protocols. Each of the one or more messages can be a variable length message and each communication channel included in the communication channels 1112 can include a stream orchestration layer that can receive the variable length message in accordance with a predefined interface, such as an interface defined using an interface description language like AsyncAPI. Each of the variable length messages can include context information that can be used to determine the route or routes for the variable length message as well as a text or binary payload of arbitrary length. Each of the routes can be configured using a polyglot stream orchestration language that is agnostic to the details of the underlying implementation of the routing tasks and destinations.

Each database included in the databases 1122 can be any kind of database that is capable of storing data and/or information and managing data and/or information. Data and/or information stored by each database can include data and/or information generated by, provided by, and/or otherwise obtained by the platform 1114. Additionally, or alternatively, data and/or information stored and/or managed by each database can include data and/or information generated by, provided by, and/or otherwise obtained by other sources such as the client devices 1110 and/or LLMs 1124 (or other ML models). One or more databases that are included in the databases 1122 can be part of a platform for storing and managing healthcare information such as electronic health records for patients, electronic records of healthcare providers, and the like, and can store and manage electronic health records for patients of healthcare providers. An example platform is the Oracle Health Millenium Platform. Additionally, one or more databases included in the databases 1122 can be provided by, managed by, and/or otherwise included as part of a cloud infrastructure of a cloud service provider (e.g., Oracle Cloud Infrastructure or OCI). Data and/or information stored and/or managed by the databases 1122 can be accessed using one or more application programming interfaces (APIs) of the databases 1122.

Each LLM included in the LLMs 1124 can be any kind of LLM that is capable of obtaining or generating or retrieving one or more results in response to one or more inputs such as one or more machine-learning prompts (hereinafter, “ML prompts” or “prompts”). ML prompts for obtaining or generating or retrieving results from the LLMs 1124 can obtained from or generated by or retrieved from or accessed from the client devices 1110, the databases 1122, the platform 1114, and/or one or more other sources such as the Internet. Each ML prompt can be configured to cause the LLMs 1124 to perform one or more tasks, which causes one or more results to be provided or generated and the like. ML prompts for the LLMs 1124 can be pre-generated (e.g., before they are needed for a particular task) and/or generated in real-time (e.g., without a delay noticeable to a human user). In some implementations, prompts for the LLMs 1124 can be engineered to achieve a desired result or results manually and/or by one or more ML models. In some implementations, ML prompts for the LLMs 1124 can be engineered on demand (i.e., in real-time and/or as needed) and/or at particular intervals (e.g., once per day, upon log in by authenticated user into the platform 1114). Each ML prompt of the one or more ML prompts can include a request, such as a query, for a task to be performed by the LLMs 1124. In some cases, an ML prompt can include additional information, such as data generated by one or more services (e.g., agent-driven services) included in the platform 1114. The additional information can include information such as one or more ML prompt templates, structured data that is configured to be interpreted (e.g., semantically interpreted) by a computing system component (e.g., an ML model, an agent-driven service, etc.), unstructured data that is configured to be interpreted (e.g., semantically interpreted) by a human, responses from one or more ML models, output data generated by one or more agent-driven services, and/or other information suitable to include in an ML prompt. LLMs included in the LLMs 1124 can be pre-trained, fine-tuned, open source, off-the-shelf, licensed, subscribed to, and the like. Additionally, LLMs included in the LLMs 1124 can include or have any size context window (e.g., can accept any number of tokens) and can be capable of interpreting complex instructions. One or more LLMs included in the LLMs 1124 can be provided by, managed by, and/or otherwise included as part of the platform 1114 and/or a cloud infrastructure of a cloud service provider (e.g., Oracle Cloud Infrastructure or OCI) that supports the platform 1114. One or more LLMs included in the LLMs 1124 can be accessed using one or more APIs of the LLMs 1124 and/or a platform hosting or supporting or providing the LLMs 1124. In some implementations, one or more additional ML models included in the environment 1100 may have one or more characteristics that are similar to characteristics described in regard to the LLMs 1124.

The platform 1114 can be configured to include various capabilities and provide various services to subscribers (e.g., end users) of the various services. In some implementations, such as in the case of an end user or subscriber being a healthcare provider, the healthcare provider can utilize the various services to facilitate the observation, care, treatment, management, and so on of their patient populations. For example, a healthcare provider can utilize the functionality provided by the various services provided by the platform 1114 to examine and/or treat and/or facilitate the examination and/or treatment of a patient; view, edit, and/or manage a patient's electronic health record; perform administrative tasks such as placing medical orders, scheduling appointments, managing patient populations, providing customer service to facilitate operation of a healthcare environment in which the healthcare provider practices, and so on.

In some implementations, the services provided by the platform 1114 can include, and are not limited to, a response engine 1115 and a knowledge engine 1117. In some implementations, one or more services provided by the platform 1114, such as the response engine 1115 and/or the knowledge engine 1117, can be configured to operate as agent-driven services, such as agent-driven services 1120. In some implementations, an execution plan guides activities of one or more of the agent-driven services 1120 provided by the platform 1114. For example, the platform 1114 can include, such as included in or in addition to the LLMs 1124, a generative AI model (or another suitable ML model included in the environment 1100) that is configured to generate (or modify) an execution plan. In this example, the execution plan can describe actions associated with one or more of the agent-driven services 1120 provided by the platform 1114. Based on the execution plan generated by the example generative AI model, one or more of the agent-driven services 1120 can be configured to operate and/or interact with one or more additional ones of the agent-driven services 1120. In some implementations, an output of the platform 1114 is described by the execution plan. For example, the example generative AI model could be configured to generate an execution plan based on request data associated with the platform 1114 (such as request data included in at least one query received by the platform 1114 from one or more of the client devices 1110). In some cases, the platform 1114 and/or the example generative AI model can determine that the request data is associated with at least one agent-driven service of the services 1120 provided by the platform 1114, such as request data associated with the response engine 1115 and/or the knowledge engine 1117. In this example, the example generative AI model can generate an execution plan that describes one or more agent tasks for the at least one agent-driven service provided by the platform 1114, such as agent tasks for generating a response to a user query and/or generating knowledge-grounded response data. For example, the response engine 1115, the knowledge engine 1117, and/or additional services in the agent-driven services 1120 generates at least one response data object, such as the knowledge-grounded response data 1190, based on a combination of multiple data outputs from the response engine 1115 and/or the knowledge engine 1117. In this example, the knowledge engine 1117 generates the knowledge-grounded response data 1190 by combining multiple data outputs from the response engine 1115 and/or the knowledge engine 1117 in a combination that is described by the execution plan.

In some implementations, the generated execution plan can omit instructions for implementing an agent task and include data outlining an agent task (e.g., data outlining one or more data sources, inputs, or requested outputs). In some implementations, one or more service of the agent-driven services 1120 can generate its own instructions for implementing an agent task based on the data outlined in the execution plan. For example, an agent-driven service included in (or otherwise associated with) the response engine 1115 can construct one or more ML prompts for generating response data, such as by using data outlined in the example execution plan to identify a prompt template (e.g., from a library of templates), a data source (e.g., a data repository storing information related to the user query 1105), and one or more of the LLMs 1124 (e.g., configured to generate text data summarizing medical information related to the user query 1105). As another example, an agent-driven service included in (or otherwise associated with) the knowledge engine 1117 can construct one or more ML prompts for generating and/or annotating the knowledge-grounded response data 1190, such as by using data outlined in the example execution plan to identify a prompt template, a data source (e.g., data summarized by the response engine 1115 and/or the data repository storing information related to the user query 1105), and one or more of the LLMs 1124 (e.g., configured to determine at least one response annotation using the data summarized by the response engine 1115). In some cases, based on the data outlined in the execution plan, the response engine 1115 and/or the knowledge engine 1117 can be configured to generate respective instructions by which the services 1116 and/or 1118 can operate and/or interact with one or more additional services provided by the platform 1114 (such as, and not limited to, additional services of the agent-driven services 1120). Examples of skill-driven and LLM-based and agent-driven digital assistants are described in U.S. patent application Ser. No. 17/648,376, filed on Jan. 19, 2022, and U.S. patent application Ser. No. 18/624,472, filed on Apr. 2, 2024, each of which are incorporated by reference as if fully set forth herein.

In the platform 1114, one or more of the agent-driven services 1120 are configured, such as based on one or more generated execution plans, to create and/or annotate one or more portions of the knowledge-grounded response data 1190. In the computing environment 1100, the agent-driven services 1120 can create the knowledge-grounded response data 1190 in response to receiving one or more queries, such as a user query 1105. To create the knowledge-grounded response data 1190, the agent-driven services 1120 perform, via the platform 1114, one or more of acquiring LLMs, execution plan creation and/or implementation, asset identification (such as identification of one or more model-selected assets 1150), and providing the knowledge-grounded response data 1190 to one or more additional computing systems, such as to the client systems 1110. For example, the platform 1114 may receive the user query 1105 from a particular one of the client systems 1110. In addition, the platform 1114 may generate at least one execution plan based on the user query 1105. In some cases, one or more of the response engine 1115, the knowledge engine 1117, or one or more additional services of the agent-driven services 1120 may identify at least one of the LLMs 1124 based on the execution plan (or respective portions of the execution plan). In addition, one or more of the response engine 1115, the knowledge engine 1117, or the one or more additional services of the agent-driven services 1120 may identify, such as from the databases 1122, at least one asset based on the execution plan (or respective portions of the execution plan). Based on the identified one(s) of the LLMs 1124 and/or the identified asset(s), one or more of the response engine 1115, the knowledge engine 1117, or the one or more additional services of the agent-driven services 1120 may generate and/or modify the knowledge-grounded response data 1190. In some cases, the knowledge-grounded response data 1190 includes a combination of response data and attention cue data, such as response data that responds to a question (or other query type) included in the user query 1105 and attention cue data that draws a user's attention to at least a portion of the response data. Examples of response data can include text data, numeric data, image data (e.g., a radiology image), tabulated data (e.g., arranged in a table or other suitable format), or other types of response data suitable for responding to a user query. Examples of attention cue data can include highlighting data (e.g., color text, color background, color-vision deficiency patterns, etc.), font data (e.g., font size, italics, bold, underlining, typeface, etc.), audio data (e.g., automatic speech generation, audible alert data, etc.), haptic data (e.g., vibration, etc.), or other suitable types of attention cue data suitable for drawing user attention to at least a portion of response data.

In some implementations, the response engine 1115 can be configured to automatically generate some or all response data that is included in the knowledge-grounded response data 1190. For example, by utilizing an execution plan that is generated based on the user query 1105, the response engine 1115 may identify a first LLM from the LLMs 1124 and one or more assets from the databases 1122, such as one or more of an asset 1150A, an asset 1150B, through an asset 1150N that are included in the model-selected assets 1150. In addition, the response engine 1115 may generate one or more ML prompts based on the one or more assets and provide the one or more ML prompts to the first LLM. Based on information received from the first LLM (e.g., in response to the one or more ML prompts), the response engine 1115 may generate response data that responds to a question included in the user query 1105. For example, if the user query 1105 includes a question “How has Ms. Henderson's new blood pressure medication been working? ” the response engine 1115 may identify, such as from an electronic health record (hereinafter, “EHR”) associated with the patient Ms. Henderson, a group of blood pressure measurements from a time period associated with a blood pressure medication currently prescribed to the patient. The response engine 1115 may select the group of blood pressure measurements as information included in the asset 1150A. In some cases, the response engine 1115 may identify one or more additional assets from the databases 1122 and include the additional assets in the model-selected assets 1150, such as including in the asset 1150B information describing the currently prescribed blood pressure medication or including in the asset 1150N information describing additional medical factors for the patient (e.g., an additional diagnosis, a preferred exercise frequency for the patient, etc.) Continuing with this example, the response engine 1115 may determine that the first LLM is fine-tuned to summarize information. In addition, the response engine 1115 may generate a first ML prompt that includes one or more of the identified assets (e.g., assets 1150A through 1150N) and provide the first ML prompt to the first LLM. Based on data received from the first LLM, e.g., data summarizing the identified assets included in the first ML prompt, the response engine 1115 may generate response data that includes a combination of text and tabulated numeric data, such as a table of blood pressure measurements and a text description of a trend in the blood pressure measurements since the patient Ms. Henderson began taking the currently prescribed blood pressure medication.

In some implementations, the knowledge engine 1117 can be configured to automatically generate some or all attention cue data that is included in the knowledge-grounded response data 1190. For example, by utilizing the execution plan that is generated based on the user query 1105, the knowledge engine 1117 may identify a second LLM from the LLMs 1124. In addition, the knowledge engine 1117 may identify one or more assets, such as one or more the response data generated by the response engine 1115 and/or one or more of the assets 1150A through 1150N. In some cases, the knowledge engine 1117 may generate one or more ML prompts based on the one or more assets and provide the one or more ML prompts to the second LLM. Based on information received from the second LLM (e.g., in response to the one or more ML prompts), the knowledge engine 1117 may generate attention cue data that draws user attention to at least a portion of the response data generated by the response engine 1115.

Continuing with the example question “How has Ms. Henderson's new blood pressure medication been working? ” the knowledge engine 1117 may determine that the second LLM is fine-tuned to identify high-relevance data in one or more assets. In addition, the knowledge engine 1117 may generate a second ML prompt that includes one or more of the identified assets (e.g., the response data generated by the response engine 1115 and the assets 1150A through 1150N) and provide the second ML prompt to the second LLM. Based on data received from the second LLM, e.g., data identifying high-relevance data included in the second ML prompt, the knowledge engine 1117 may generate attention cue data that draws attention to the high-relevance data, such as color highlighting that draws attention to a trend in the blood pressure measurements and a bold font style that draws attention to information describing a possible interaction of the currently prescribed blood pressure medication with an additional medication frequently prescribed for an additional diagnosis of the patient. In addition, the knowledge engine 1117 may generate or modify the knowledge-grounded response data 1190 to include the attention cue data, e.g., modifying the knowledge-grounded response data 1190 to apply the color highlighting to at least a portion of the tabulated numeric data and the bold font style to at least a portion of the text data. In some cases, the knowledge engine 1117 may modify the knowledge-grounded response data 1190 to include additional response data, such as interactive reference data (e.g., a URL address) that provides one or more references describing a source of information included in the knowledge-grounded response data 1190. Continuing with the above example, the knowledge engine 1117 may generate first interactive reference data that provides a first reference to one or more EHRs including blood pressure measurements for the patient. In addition, the knowledge engine 1117 may generate second interactive reference data that provides a second reference to medication information (e.g., a medication reference database) describing possible interactions of the currently prescribed blood pressure medication.

In some implementations, the platform 1114 (or a component thereof) may provide the knowledge-grounded response data 1190 to one or more additional computing systems. For example, the platform 1114 may identify a particular client device of the client devices 1110 from which the user query 1105 was received. In addition, the platform 1114 may provide the knowledge-grounded response data 1190 to the particular client device. In some cases, the particular client device is configured to perform one or more operations based on the knowledge-grounded response data 1190, such as operations related to displaying the combination of the response data and the attention cue data included in the knowledge-grounded response data 1190. For example, the particular client device may be configured to display the response data as annotated by the attention cue data, e.g., the table of blood pressure measurements and the text description as annotated by the color highlighting, the bold font style, and the interactive reference data. In addition, the particular client device may be configured to receive additional input data based on the knowledge-grounded response data 1190, such as a user input indicating a selection of at least a portion of the knowledge-grounded response data 1190. For example, responsive to receiving a user selection input of the first interactive reference data, the particular client device may be configured to send, to the platform 1114, a request to receive at least a portion of the first reference, such as the one or more EHRs (or a portion thereof) including blood pressure measurements for the patient. In addition, responsive to receiving an additional user selection input of the second interactive reference data, the particular client device may be configured to send, to the platform 1114, an additional request to receive at least a portion of the second reference, such as the medication information (or a portion thereof) describing possible interactions of the currently prescribed blood pressure medication. In some cases, the data architecture and/or configuration of the platform 1114, such as the combination of the response engine 1115 and the knowledge engine 1117 and/or combination of some or all described features thereof, can improve user comprehension of information provided in response to user queries, such as the user query 1105. For example, the combination of the response data with the attention cue data, such as included in the knowledge-grounded response data 1190, can improve comprehension or reduce reading time by a user, such as by drawing the user's attention to portions of the response data that are annotated by the attention cue data. In addition, the combination of the response data with the interactive reference data that is included in the attention cue data, such as included in the knowledge-grounded response data 1190, can improve user trust in the response data by facilitating fast identification of potentially inaccurate data (e.g., hallucinations) generated by one or more of the LLMs 1124, such as by providing fast access to reference information via the interactive reference data.

In FIG. 11, the response engine 1115 and the knowledge engine 1117 are described as utilizing a particular execution plan (e.g., respective portions of a same execution plan), and other implementations are possible. For example, a cloud service provider platform may generate a respective execution plan for each particular agent-driven service that is included in (or otherwise utilized by) the example cloud service provider platform. In FIG. 11, the response engine 1115 and the knowledge engine 1117 are described as respectively identifying the first LLM and the second LLM from the LLMs 1124, and other implementations are possible. For example, in various instances, the response engine 1115 and the knowledge engine 1117 (or others of the agent-driven services 1120) may identify from the LLMs 1124 at least one same LLM, at least one different LLM, and/or a combination of different and same LLMs.

In some instances, the agent-driven services 1120 can be utilized to access pre-trained and/or fine-tuned ML models, such as one or more of the LLMs 1124. The pre-trained ML models serve as foundational elements, possessing extensive language understanding derived from vast datasets. This capability enables the models to generate coherent responses across various topics, facilitating transfer learning. Pre-trained models offer cost-effectiveness and flexibility, which allows for scalable improvements and continuous pre-training with new data, often establishing benchmarks in natural language processing tasks. Conversely, fine-tuned models are specifically trained for tasks or industries (e.g., plan creation utilizing the LLM's in-context learning capability, knowledge or information retrieval on behalf of an agent, response generation for human-like conversation, etc.), enhancing their performance on specific applications and enabling efficient learning from smaller, specialized datasets. Fine-tuning provides advantages such as task specialization, data efficiency, quicker training times, model customization, and resource efficiency. In some embodiments, fine-tuning may be particularly advantageous for niche applications and ongoing enhancement. In other instances, the agent-driven services 1120 can be utilized to pre-train and/or fine-tune the LLMs. The agent-driven services 1120, or any subset thereof, may be standalone or part of a machine-learning operationalization framework, inclusive of hardware components like processors (e.g., CPU, GPU, TPU, FPGA, or any combination), memory, and storage. This framework operates software or computer program instructions (e.g., TensorFlow, PyTorch, Keras, etc.) to execute arithmetic, logic, input/output commands for training, validating, and deploying machine-learning models in a production environment. In certain instances, the agent-driven services 1120 implement the training, validating, and deploying of the models using a cloud platform such as Oracle Cloud Infrastructure (OCI). In some cases, leveraging a cloud platform can make machine-learning more accessible, flexible, and cost-effective, which can facilitate faster model development and deployment for developers.

Although not shown, the platform 1114 can include other capabilities and services such as authentication services, management services, task management services, notification services, and the like. The various capabilities and services of the platform 1114 can be implemented utilizing one or more computing resources and/or servers of the platform 1114 and provided by the platform 1114 by way of subscriptions. Additionally, or alternatively, while FIG. 11 shows the services of the platform 1114 as being separate services, one or more of the services can be combined with other services and/or be considered to be a sub-service of another service. In some implementations, a particular service in the agent-driven services 1120 may utilize an output from another service in the agent-driven services 1120, such as to facilitate quick completion of one or more operations by the particular service. For example, as shown in FIG. 8, one or more of the response engine 1115 or the knowledge engine 1117 can include at least one sub-service. In addition, one or more of the response engine 1115 or the knowledge engine 1117 can access one or more outputs from one or more services of the agent-driven services 1120, such as agent output data 1130. In some cases, the availability of the agent output data 1130 to multiple services in the agent-driven services 1120, such as at least the response engine 1115 and the knowledge engine 1117, can improve response time by the multiple services in the agent-driven services 1120. For example, the response engine 1115 and the knowledge engine 1117 may provide one or more outputs with decreased response time and decreased use of computing resources (e.g., processing resources, memory resources, ML model resources, etc.) by using some or all of the agent output data 1130 as input data.

FIG. 11 depicts the structured data 1132 and the unstructured data 1134 as being included in the agent output data 1130, and other implementations are possible, such as one or more of the data 1132 or 1134 being included in the model-selected assets 1150. In some cases, one or more of the structured data 1132 and the unstructured data 1134 is an output from one or more additional services of the agent-driven services 1120, such as additional services configured for generating text data based on audio data (e.g., transcription of spoken conversation between a patient and a healthcare provider), identifying EHRs for a particular patient, or other tasks suitable to be performed by the agent-driven services 1120. In FIG. 11, the structured data 1132 can include data that is structured to be interpreted by a computing device, such as database records, JavaScript data objects, or other data objects (e.g., included in one or more EHRs) that are intended for computer interpretation (e.g., not intended for human interpretation). In FIG. 11, the unstructured data 1134 can include data that lacks a structure interpreted by a computing device, such as patient appointment notes (e.g., Subjective, Objective, Assessment, and Plan notes, “SOAP notes”), medical reference materials (e.g., medication documentation, surgical procedure guidelines, etc.), or other types of information that are intended for human interpretation.

In the computing environment 1100, the response engine 1115 can include at least one sub-service, such as a response data prioritization service 1116. In some cases, the response data prioritization service 1116 may access at least one of the agent output data 1130, such as one or more of the structured data 1132 or the unstructured data 1134. In addition, the response data prioritization service 1116 may evaluate portions of the structured data 1132 or the unstructured data 1134 for potential inclusion in response data. For example, the response data prioritization service 1116 may evaluate a data portion to determine whether the data is relevant to a question or other information in the user query 1105. In some cases, the response data prioritization service 1116 may send one or more portions of the structured data 1132 or the unstructured data 1134 to at least one LLM of the LLMs 1124, such as in an ML prompt. In some implementations, the ML prompt also includes data corresponding to the user query 1105, such as a question included in the user query 1105 or additional data (e.g., determined by a service in the agent-driven services 1120), such as additional data corresponding to the user query 1105.

In the computing environment 1100, the response data prioritization service 1116 may receive, from the at least one LLM, multiple data portions that are extracted from one or more of the structured data 1132 or the unstructured data 1134. In addition, the response data prioritization service 1116 may determine a relevance status for each of the multiple data portions, such as by comparing each data portion to one or more relevance threshold values. In some cases, the response data prioritization service 1116 may determine, such as by determining a similarity (e.g., semantic similarity) between each data portion and query data (e.g., information associated with the user query 1105), whether each data portion exceeds (or fulfill another relationship with) the one or more relevance threshold values. For example, based on a comparison to a first relevance threshold value, the response data prioritization service 1116 may identify various data portions as high-relevance data, such as high-relevance data that directly answers one or more questions included in the user query 1105. In some cases, the response data prioritization service 1116 may select one or more of the LLMs 1124 to modify some of the high-relevance data before inclusion in response data 1136. For example, the response data prioritization service 1116 may provide a first portion of the high-relevance data to a first LLM (e.g., from the LLMs 1124) that is fine-tuned to summarize received information in text summary data. Examples of text summary data can include paragraphs, single sentences, or other human-readable text data that summarizes larger amounts of information. In addition, the response data prioritization service 1116 may modify the response data 1136 to include the text summary data which summarizes the high-relevance data. As another example, the response data prioritization service 1116 may provide a second portion of the high-relevance data to a second LLM (e.g., from the LLMs 1124) that is fine-tuned to arrange received information as tabulated data, such as multiple items of information that are intended to be interpreted as a group. Examples of tabulated data can include tables, bulleted lists, numbered lists, or other organized arrangements of multiple items of information intended to be interpreted as a group. Examples of information that could be arranged as tabulated data can include a group of lab results, a group of comparison medications (e.g., generics, non-generics, etc.), a group of potential side effects of a surgical procedure, or other groups of information items. In addition, the response data prioritization service 1116 may modify the response data 1136 to include the tabulated data in which the high-relevance data is arranged.

In some implementations, based on a comparison to a second relevance threshold value, the response data prioritization service 1116 may identify one or more data portions (e.g., extracted from one or more of the structured data 1132 or the unstructured data 1134) as medium-relevance data. In some cases, the medium-relevance data can include supplemental data that does not directly answer one or more questions included in the user query 1105 and which provides additional data about a topic identified in the one or more questions. Examples of supplemental data can include information about a diagnosed condition, information about a patient circumstance (e.g., a high-exercise lifestyle, a preference to avoid injected medications, etc.), or other types of information that are generally related to a question. In some cases, the response data prioritization service 1116 may select one or more of the LLMs 1124 to modify some of the medium-relevance data before inclusion in the response data 1136. For example, the response data prioritization service 1116 may provide a portion of the medium-relevance data to the first LLM that is fine-tuned to summarize received information in text summary data. In addition, the response data prioritization service 1116 may modify the response data 936 to include additional text summary data which summarizes the medium-relevance data.

In the computing environment 1100, the knowledge engine 1117 can include at least one sub-service, such as one or more of an annotation selection service 1118 or a display preparation service 1119. In some cases, one or more of the annotation selection service 1118 or the display preparation service 1119 may access at least one of the agent output data 1130, such as the response data 1136 that is generated by the response engine 1115. Examples of computer-implemented instructions related to display preparation service can include hypertext markup language (HTML) instructions, extensible markup language (XML) instructions, or other suitable types of instructions for implementing data display (e.g., visual display, audio display, etc.) via one or more user interface devices.

In the computing environment 1100, the annotation selection service 1118 may provide one or more portions of the response data 1136 to at least one LLM of the LLMs 1124, such as in an ML prompt. In some implementations, the ML prompt also includes additional data (e.g., determined by a service in the agent-driven services 1120), such as additional data corresponding to one or more of the user query 1105, the structured data 1132, or the unstructured data 1134.

For example, the at least one LLM may be fine-tuned to identify, in the response data 1136, one or more portions of data that have a relatively high similarity to data included in one or more of the user query 1105, the structured data 1132, or the unstructured data 1134, such as a portion of high-relevance text summary data in the response data 1136 that has a high similarity to text data of a question included in the user query 1105. In some cases, the annotation selection service 1118 may receive, from the at least one LLM, data identifying at least one portion of the response data 1136 for annotation. In addition, the annotation selection service 1118 may determine one or more types of annotations to apply to the identified portion of the response data 1136, such as annotations for highlighting, font styles, or other types of annotations that can be applied to response data. In some implementations, the annotation selection service 1118 may determine at least one type of annotation that applies interactive reference data to the identified portion of the response data 1136. For example, the annotation selection service 1118 may determine one or more sources for the identified portion of the response data 1136, such as a source document and/or source database associated with one or more of the structured data 1132 or the unstructured data 1134. Based on the determined one or more sources, the annotation selection service 1118 may generate interactive reference data that indicates the source(s) for the identified portion of the response data 1136. In some cases, the annotation selection service 1118 may identify source address data associated with the source(s) for the identified portion of the response data 1136. Examples of source address data can include a network address (e.g., a URL, a MAC address), computing component identification data (e.g., identification of a particular database, etc.), document identification data (e.g., identification of a particular document, identification of a section within a document, etc.), or other types of address data that can identify a location (or other identification type) for a source repository.

In the computing environment 1100, the display preparation service 1119 may identify one or more associated portions of response data. In addition, the display preparation service 1119 may generate one or more computer-implemented instructions that combine the associated portions of response data for presentation via one or more user interface devices. In addition, the display preparation service 1119 may generate at least one computer-implemented instruction that combines the associated portions, such as an HTML instruction (or other suitable instruction type) that applies a bold typeface to the sentence. As another example, the display preparation service 1119 may determine an additional association between a second portion of the response data 1136 that indicates tabulated data, such as a set of blood pressure measurements, and an additional annotation including interactive reference data, such as a patient chart that is a source document for the set of blood pressure measurements. In addition, the display preparation service 1119 may generate at least one additional computer-implemented instruction that combines the additional associated portions, such as an additional HTML instruction (or other suitable instruction type) that applies an interactive link to the tabulated set of blood pressure measurements, e.g., the interactive link is directed to the patient chart. The computing environment 1100 depicted in FIG. 11 is merely exemplary and not intended to unduly limit the scope of claimed embodiments. One of ordinary skill in the art would recognize many possible variations, alternatives, and modifications. For example, in some implementations, the computing environment 1100 900 can be implemented using more or fewer services than those shown in FIG. 11, may combine two or more services, or may have a different configuration or arrangement of services.

### Examples of Cloud Infrastructure Architectures

The term cloud service is generally used to refer to a service that is made available by a cloud service provider (CSP) to users (e.g., cloud service customers) on demand (e.g., via a subscription model) using systems and infrastructure (cloud infrastructure) provided by the CSP. Typically, the servers and systems that make up the CSP's infrastructure are separate from the user's own on-premise servers and systems. Users can thus avail themselves of cloud services provided by the CSP without having to purchase separate hardware and software resources for the services. Cloud services are designed to provide a subscribing user easy, scalable access to applications and computing resources without the user having to invest in procuring the infrastructure that is used for providing the services.

There are several cloud service providers that offer various types of cloud services. As discussed herein, there are various types or models of cloud services including IaaS, software as a service (SaaS), platform as a service (PaaS), and others. A user can subscribe to one or more cloud services provided by a CSP. The user can be any entity such as an individual, an organization, an enterprise, and the like. When a user subscribes to or registers for a service provided by a CSP, a tenancy or an account is created for that user. The user can then, via this account, access the subscribed-to one or more cloud resources associated with the account.

As noted above, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

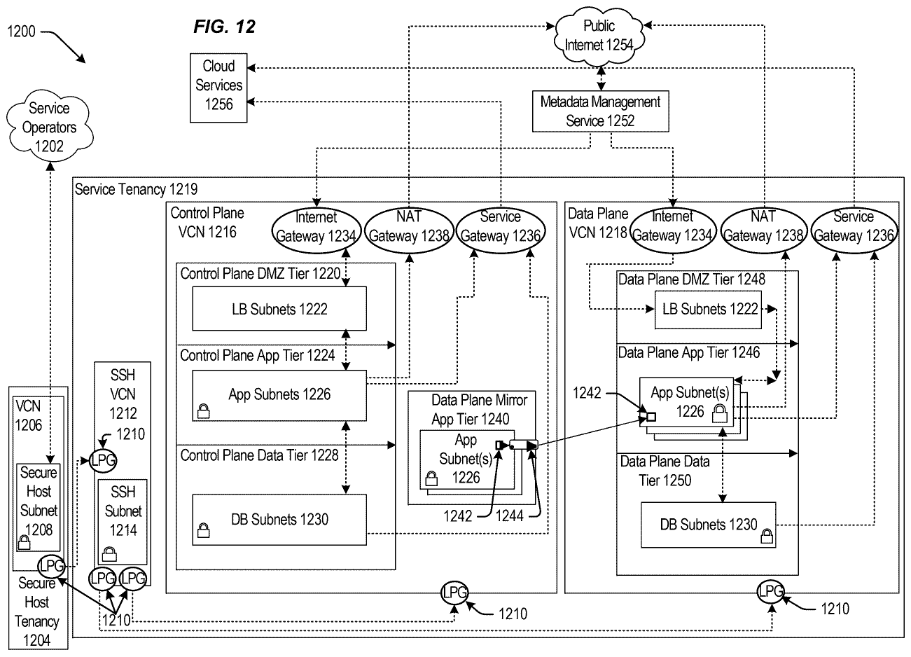

FIG. 12 is a block diagram 1200 illustrating an example pattern of an IaaS architecture, according to at least one embodiment. Service operators 1202 can be communicatively coupled to a secure host tenancy 1204 that can include a virtual cloud network (VCN) 1206 and a secure host subnet 1208. In some examples, the service operators 1202 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1206 and/or the Internet.

The VCN 1206 can include a local peering gateway (LPG) 1210 that can be communicatively coupled to a secure shell (SSH) VCN 1212 via an LPG 1210 contained in the SSH VCN 1212. The SSH VCN 1212 can include an SSH subnet 1214, and the SSH VCN 1212 can be communicatively coupled to a control plane VCN 1216 via the LPG 1210 contained in the control plane VCN 1216. Also, the SSH VCN 1212 can be communicatively coupled to a data plane VCN 1218 via an LPG 1210. The control plane VCN 1216 and the data plane VCN 1218 can be contained in a service tenancy 1219 that can be owned and/or operated by the IaaS provider.

The control plane VCN 1216 can include a control plane demilitarized zone (DMZ) tier 1220 that acts as a perimeter network (e.g., portions of a corporate network between the corporate intranet and external networks). The DMZ-based servers may have restricted responsibilities and help keep breaches contained. Additionally, the DMZ tier 1220 can include one or more load balancer (LB) subnet(s) 1222, a control plane app tier 1224 that can include app subnet(s) 1226, a control plane data tier 1228 that can include database (DB) subnet(s) 1230 (e.g., frontend DB subnet(s) and/or backend DB subnet(s)). The LB subnet(s) 1222 contained in the control plane DMZ tier 1220 can be communicatively coupled to the app subnet(s) 1226 contained in the control plane app tier 1224 and an Internet gateway 1234 that can be contained in the control plane VCN 1216, and the app subnet(s) 1226 can be communicatively coupled to the DB subnet(s) 1230 contained in the control plane data tier 1228 and a service gateway 1236 and a network address translation (NAT) gateway 1238. The control plane VCN 1216 can include the service gateway 1236 and the NAT gateway 1238.

The control plane VCN 1216 can include a data plane mirror app tier 1240 that can include app subnet(s) 1226. The app subnet(s) 1226 contained in the data plane mirror app tier 1240 can include a virtual network interface controller (VNIC) 1242 that can execute a compute instance 1244. The compute instance 1244 can communicatively couple the app subnet(s) 1226 of the data plane mirror app tier 1240 to app subnet(s) 1226 that can be contained in a data plane app tier 1246.

The data plane VCN 1218 can include the data plane app tier 1246, a data plane DMZ tier 1248, and a data plane data tier 1250. The data plane DMZ tier 1248 can include LB subnet(s) 1222 that can be communicatively coupled to the app subnet(s) 1226 of the data plane app tier 1246 and the Internet gateway 1234 of the data plane VCN 1218. The app subnet(s) 1226 can be communicatively coupled to the service gateway 1236 of the data plane VCN 1218 and the NAT gateway 1238 of the data plane VCN 1218. The data plane data tier 1250 can also include the DB subnet(s) 1230 that can be communicatively coupled to the app subnet(s) 1226 of the data plane app tier 1246.

The Internet gateway 1234 of the control plane VCN 1216 and of the data plane VCN 1218 can be communicatively coupled to a metadata management service 1252 that can be communicatively coupled to public Internet 1254. Public Internet 1254 can be communicatively coupled to the NAT gateway 1238 of the control plane VCN 1216 and of the data plane VCN 1218. The service gateway 1236 of the control plane VCN 1216 and of the data plane VCN 1218 can be communicatively coupled to cloud services 1256.

In some examples, the service gateway 1236 of the control plane VCN 1216 or of the data plane VCN 1218 can make application programming interface (API) calls to cloud services 1256 without going through public Internet 1254. The API calls to cloud services 1256 from the service gateway 1236 can be one-way: the service gateway 1236 can make API calls to cloud services 1256, and cloud services 1256 can send requested data to the service gateway 1236. But, cloud services 1256 may not initiate API calls to the service gateway 1236.

In some examples, the secure host tenancy 1204 can be directly connected to the service tenancy 1219, which may be otherwise isolated. The secure host subnet 1208 can communicate with the SSH subnet 1214 through an LPG 1210 that may enable two-way communication over an otherwise isolated system. Connecting the secure host subnet 1208 to the SSH subnet 1214 may give the secure host subnet 1208 access to other entities within the service tenancy 1219.

The control plane VCN 1216 may allow users of the service tenancy 1219 to set up or otherwise provision desired resources. Desired resources provisioned in the control plane VCN 1216 may be deployed or otherwise used in the data plane VCN 1218. In some examples, the control plane VCN 1216 can be isolated from the data plane VCN 1218, and the data plane mirror app tier 1240 of the control plane VCN 1216 can communicate with the data plane app tier 1246 of the data plane VCN 1218 via VNICs 1242 that can be contained in the data plane mirror app tier 1240 and the data plane app tier 1246.

In some examples, users of the system, or customers, can make requests, for example create, read, update, or delete (CRUD) operations, through public Internet 1254 that can communicate the requests to the metadata management service 1252. The metadata management service 1252 can communicate the request to the control plane VCN 1216 through the Internet gateway 1234. The request can be received by the LB subnet(s) 1222 contained in the control plane DMZ tier 1220. The LB subnet(s) 1222 may determine that the request is valid, and in response to this determination, the LB subnet(s) 1222 can transmit the request to app subnet(s) 1226 contained in the control plane app tier 1224. If the request is validated and requires a call to public Internet 1254, the call to public Internet 1254 may be transmitted to the NAT gateway 1238 that can make the call to public Internet 1254. Metadata that may be desired to be stored by the request can be stored in the DB subnet(s) 1230.

In some examples, the data plane mirror app tier 1240 can facilitate direct communication between the control plane VCN 1216 and the data plane VCN 1218. For example, changes, updates, or other suitable modifications to configuration may be desired to be applied to the resources contained in the data plane VCN 1218. Via a VNIC 1242, the control plane VCN 1216 can directly communicate with, and can thereby execute the changes, updates, or other suitable modifications to configuration to, resources contained in the data plane VCN 1218.

In some embodiments, the control plane VCN 1216 and the data plane VCN 1218 can be contained in the service tenancy 1219. In this case, the user, or the customer, of the system may not own or operate either the control plane VCN 1216 or the data plane VCN 1218. Instead, the IaaS provider may own or operate the control plane VCN 1216 and the data plane VCN 1218, both of which may be contained in the service tenancy 1219. This embodiment can enable isolation of networks that may prevent users or customers from interacting with other users', or other customers', resources. Also, this embodiment may allow users or customers of the system to store databases privately without needing to rely on public Internet 1254, which may not have a desired level of threat prevention, for storage.

In other embodiments, the LB subnet(s) 1222 contained in the control plane VCN 1216 can be configured to receive a signal from the service gateway 1236. In this embodiment, the control plane VCN 1216 and the data plane VCN 1218 may be configured to be called by a customer of the IaaS provider without calling public Internet 1254. Customers of the IaaS provider may desire this embodiment since database(s) that the customers use may be controlled by the IaaS provider and may be stored on the service tenancy 1219, which may be isolated from public Internet 1254.

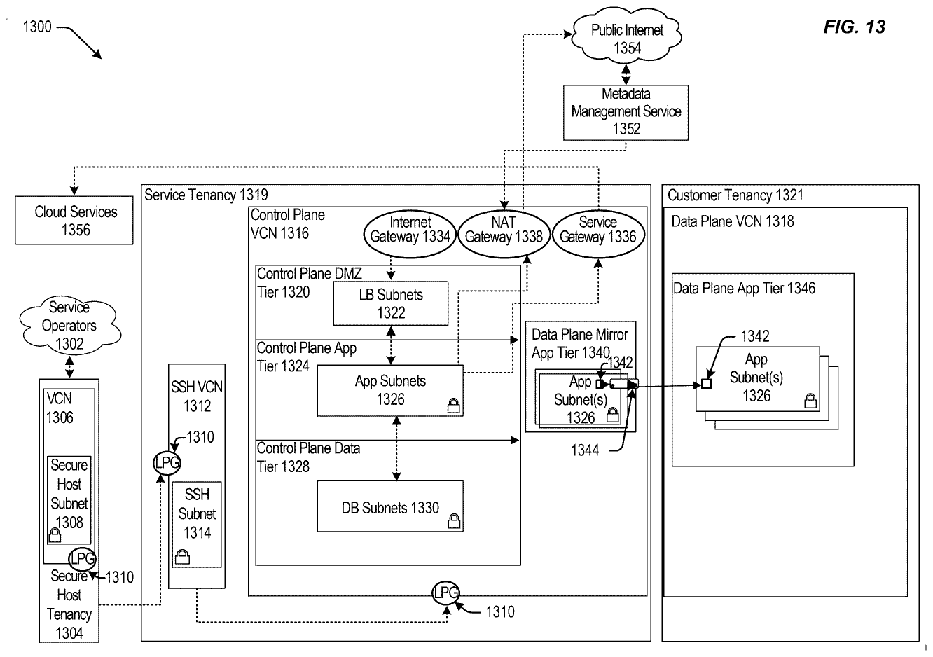

FIG. 13 is a block diagram 1300 illustrating another example pattern of an IaaS architecture, according to at least one embodiment. Service operators 1302 (e.g., service operators 1202 of FIG. 12) can be communicatively coupled to a secure host tenancy 1304 (e.g., the secure host tenancy 1204 of FIG. 12) that can include a virtual cloud network (VCN) 1306 (e.g., the VCN 1206 of FIG. 12) and a secure host subnet 1308 (e.g., the secure host subnet 1208 of FIG. 12). The VCN 1306 can include a local peering gateway (LPG) 1310 (e.g., the LPG 1210 of FIG. 12) that can be communicatively coupled to a secure shell (SSH) VCN 1312 (e.g., the SSH VCN 1212 of FIG. 12) via an LPG 1210 contained in the SSH VCN 1312. The SSH VCN 1312 can include an SSH subnet 1314 (e.g., the SSH subnet 1214 of FIG. 12), and the SSH VCN 1312 can be communicatively coupled to a control plane VCN 1316 (e.g., the control plane VCN 1216 of FIG. 12) via an LPG 1310 contained in the control plane VCN 1316. The control plane VCN 1316 can be contained in a service tenancy 1319 (e.g., the service tenancy 1219 of FIG. 12), and the data plane VCN 1318 (e.g., the data plane VCN 1218 of FIG. 12) can be contained in a customer tenancy 1321 that may be owned or operated by users, or customers, of the system.

The control plane VCN 1316 can include a control plane DMZ tier 1320 (e.g., the control plane DMZ tier 1220 of FIG. 12) that can include LB subnet(s) 1322 (e.g., LB subnet(s) 1222 of FIG. 12), a control plane app tier 1324 (e.g., the control plane app tier 1224 of FIG. 12) that can include app subnet(s) 1326 (e.g., app subnet(s) 1226 of FIG. 12), a control plane data tier 1328 (e.g., the control plane data tier 1228 of FIG. 12) that can include database (DB) subnet(s) 1330 (e.g., similar to DB subnet(s) 1230 of FIG. 12). The LB subnet(s) 1322 contained in the control plane DMZ tier 1320 can be communicatively coupled to the app subnet(s) 1326 contained in the control plane app tier 1324 and an Internet gateway 1334 (e.g., the Internet gateway 1234 of FIG. 12) that can be contained in the control plane VCN 1316, and the app subnet(s) 1326 can be communicatively coupled to the DB subnet(s) 1330 contained in the control plane data tier 1328 and a service gateway 1336 (e.g., the service gateway 1236 of FIG. 12) and a network address translation (NAT) gateway 1338 (e.g., the NAT gateway 1238 of FIG. 12). The control plane VCN 1316 can include the service gateway 1336 and the NAT gateway 1338.

The control plane VCN 1316 can include a data plane mirror app tier 1340 (e.g., the data plane mirror app tier 1240 of FIG. 12) that can include app subnet(s) 1326. The app subnet(s) 1326 contained in the data plane mirror app tier 1340 can include a virtual network interface controller (VNIC) 1342 (e.g., the VNIC of 1242) that can execute a compute instance 1344 (e.g., similar to the compute instance 1244 of FIG. 12). The compute instance 1344 can facilitate communication between the app subnet(s) 1326 of the data plane mirror app tier 1340 and the app subnet(s) 1326 that can be contained in a data plane app tier 1346 (e.g., the data plane app tier 1246 of FIG. 12) via the VNIC 1342 contained in the data plane mirror app tier 1340 and the VNIC 1342 contained in the data plane app tier 1346.

The Internet gateway 1334 contained in the control plane VCN 1316 can be communicatively coupled to a metadata management service 1352 (e.g., the metadata management service 1252 of FIG. 12) that can be communicatively coupled to public Internet 1354 (e.g., public Internet 1254 of FIG. 12). Public Internet 1354 can be communicatively coupled to the NAT gateway 1338 contained in the control plane VCN 1316. The service gateway 1336 contained in the control plane VCN 1316 can be communicatively coupled to cloud services 1356 (e.g., cloud services 1256 of FIG. 12).

In some examples, the data plane VCN 1318 can be contained in the customer tenancy 1321. In this case, the IaaS provider may provide the control plane VCN 1316 for each customer, and the IaaS provider may, for each customer, set up a unique compute instance 1344 that is contained in the service tenancy 1319. Each compute instance 1344 may allow communication between the control plane VCN 1316, contained in the service tenancy 1319, and the data plane VCN 1318 that is contained in the customer tenancy 1321. The compute instance 1344 may allow resources, that are provisioned in the control plane VCN 1316 that is contained in the service tenancy 1319, to be deployed or otherwise used in the data plane VCN 1318 that is contained in the customer tenancy 1321.

In other examples, the customer of the IaaS provider may have databases that live in the customer tenancy 1321. In this example, the control plane VCN 1316 can include the data plane mirror app tier 1340 that can include app subnet(s) 1326. The data plane mirror app tier 1340 can reside in the data plane VCN 1318, but the data plane mirror app tier 1340 may not live in the data plane VCN 1318. That is, the data plane mirror app tier 1340 may have access to the customer tenancy 1321, but the data plane mirror app tier 1340 may not exist in the data plane VCN 1318 or be owned or operated by the customer of the IaaS provider. The data plane mirror app tier 1340 may be configured to make calls to the data plane VCN 1318 but may not be configured to make calls to any entity contained in the control plane VCN 1316. The customer may desire to deploy or otherwise use resources in the data plane VCN 1318 that are provisioned in the control plane VCN 1316, and the data plane mirror app tier 1340 can facilitate the desired deployment, or other usage of resources, of the customer.

In some embodiments, the customer of the IaaS provider can apply filters to the data plane VCN 1318. In this embodiment, the customer can determine what the data plane VCN 1318 can access, and the customer may restrict access to public Internet 1354 from the data plane VCN 1318. The IaaS provider may not be able to apply filters or otherwise control access of the data plane VCN 1318 to any outside networks or databases. Applying filters and controls by the customer onto the data plane VCN 1318, contained in the customer tenancy 1321, can help isolate the data plane VCN 1318 from other customers and from public Internet 1354.

In some embodiments, cloud services 1356 can be called by the service gateway 1336 to access services that may not exist on public Internet 1354, on the control plane VCN 1316, or on the data plane VCN 1318. The connection between cloud services 1356 and the control plane VCN 1316 or the data plane VCN 1318 may not be live or continuous. Cloud services 1356 may exist on a different network owned or operated by the IaaS provider. Cloud services 1356 may be configured to receive calls from the service gateway 1336 and may be configured to not receive calls from public Internet 1354. Some cloud services 1356 may be isolated from other cloud services 1356, and the control plane VCN 1316 may be isolated from cloud services 1356 that may not be in the same region as the control plane VCN 1316. For example, the control plane VCN 1316 may be located in “Region 1,” and cloud service “Deployment 12,” may be located in Region 1 and in “Region 2.” If a call to Deployment 12 is made by the service gateway 1336 contained in the control plane VCN 1316 located in Region 1, the call may be transmitted to Deployment 12 in Region 1. In this example, the control plane VCN 1316, or Deployment 12 in Region 1, may not be communicatively coupled to, or otherwise in communication with, Deployment 12 in Region 2.

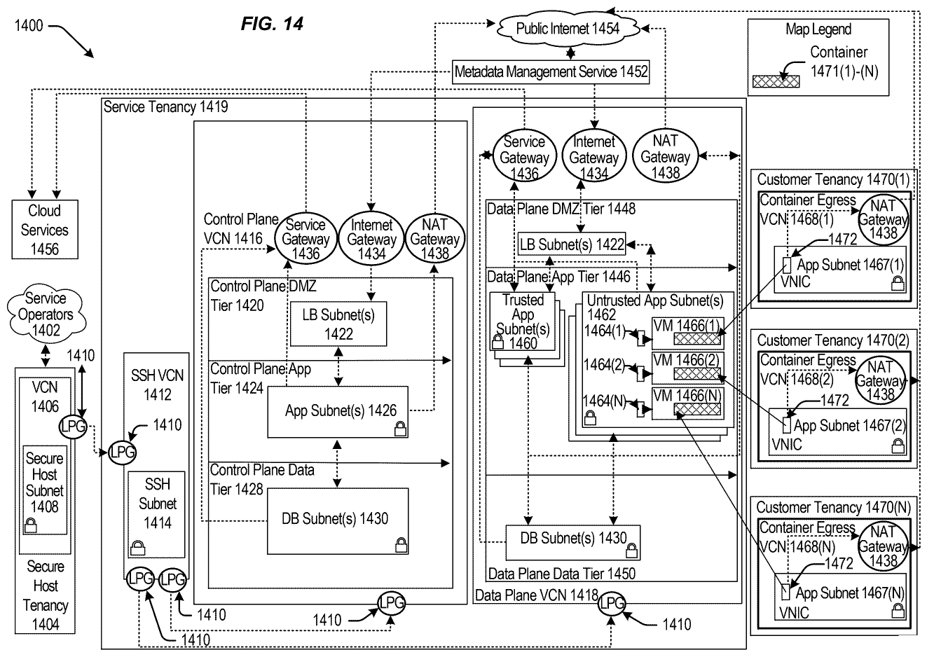

FIG. 14 is a block diagram 1400 illustrating another example pattern of an IaaS architecture, according to at least one embodiment. Service operators 1402 (e.g., service operators 1202 of FIG. 12) can be communicatively coupled to a secure host tenancy 1404 (e.g., the secure host tenancy 1204 of FIG. 12) that can include a virtual cloud network (VCN) 1406 (e.g., the VCN 1206 of FIG. 12) and a secure host subnet 1408 (e.g., the secure host subnet 1208 of FIG. 12). The VCN 1406 can include an LPG 1410 (e.g., the LPG 1210 of FIG. 12) that can be communicatively coupled to an SSH VCN 1412 (e.g., the SSH VCN 1212 of FIG. 12) via an LPG 1410 contained in the SSH VCN 1412. The SSH VCN 1412 can include an SSH subnet 1414 (e.g., the SSH subnet 1214 of FIG. 12), and the SSH VCN 1412 can be communicatively coupled to a control plane VCN 1416 (e.g., the control plane VCN 1216 of FIG. 12) via an LPG 1410 contained in the control plane VCN 1416 and to a data plane VCN 1418 (e.g., the data plane 1218 of FIG. 12) via an LPG 1410 contained in the data plane VCN 1418. The control plane VCN 1416 and the data plane VCN 1418 can be contained in a service tenancy 1419 (e.g., the service tenancy 1219 of FIG. 12).

The control plane VCN 1416 can include a control plane DMZ tier 1420 (e.g., the control plane DMZ tier 1220 of FIG. 12) that can include load balancer (LB) subnet(s) 1422 (e.g., LB subnet(s) 1222 of FIG. 12), a control plane app tier 1424 (e.g., the control plane app tier 1224 of FIG. 12) that can include app subnet(s) 1426 (e.g., similar to app subnet(s) 1226 of FIG. 12), a control plane data tier 1428 (e.g., the control plane data tier 1228 of FIG. 12) that can include DB subnet(s) 1430. The LB subnet(s) 1422 contained in the control plane DMZ tier 1420 can be communicatively coupled to the app subnet(s) 1426 contained in the control plane app tier 1424 and to an Internet gateway 1434 (e.g., the Internet gateway 1234 of FIG. 12) that can be contained in the control plane VCN 1416, and the app subnet(s) 1426 can be communicatively coupled to the DB subnet(s) 1430 contained in the control plane data tier 1428 and to a service gateway 1436 (e.g., the service gateway of FIG. 12) and a network address translation (NAT) gateway 1438 (e.g., the NAT gateway 1238 of FIG. 12). The control plane VCN 1416 can include the service gateway 1436 and the NAT gateway 1438.

The data plane VCN 1418 can include a data plane app tier 1446 (e.g., the data plane app tier 1246 of FIG. 12), a data plane DMZ tier 1448 (e.g., the data plane DMZ tier 1248 of FIG. 12), and a data plane data tier 1450 (e.g., the data plane data tier 1250 of FIG. 12). The data plane DMZ tier 1448 can include LB subnet(s) 1422 that can be communicatively coupled to trusted app subnet(s) 1460 and untrusted app subnet(s) 1462 of the data plane app tier 1446 and the Internet gateway 1434 contained in the data plane VCN 1418. The trusted app subnet(s) 1460 can be communicatively coupled to the service gateway 1436 contained in the data plane VCN 1418, the NAT gateway 1438 contained in the data plane VCN 1418, and DB subnet(s) 1430 contained in the data plane data tier 1450. The untrusted app subnet(s) 1462 can be communicatively coupled to the service gateway 1436 contained in the data plane VCN 1418 and DB subnet(s) 1430 contained in the data plane data tier 1450. The data plane data tier 1450 can include DB subnet(s) 1430 that can be communicatively coupled to the service gateway 1436 contained in the data plane VCN 1418.

The untrusted app subnet(s) 1462 can include one or more primary VNICs 1464(1)-(N) that can be communicatively coupled to tenant virtual machines (VMs) 1466(1)-(N). Each tenant VM 1466(1)-(N) can be communicatively coupled to a respective app subnet 1467(1)-(N) that can be contained in respective container egress VCNs 1468(1)-(N) that can be contained in respective customer tenancies 1470(1)-(N). Respective secondary VNICs 1472(1)-(N) can facilitate communication between the untrusted app subnet(s) 1462 contained in the data plane VCN 1418 and the app subnet contained in the container egress VCNs 1468(1)-(N). Each container egress VCNs 1468(1)-(N) can include a NAT gateway 1438 that can be communicatively coupled to public Internet 1454 (e.g., public Internet 1254 of FIG. 12).

The Internet gateway 1434 contained in the control plane VCN 1416 and contained in the data plane VCN 1418 can be communicatively coupled to a metadata management service 1452 (e.g., the metadata management system 1252 of FIG. 12) that can be communicatively coupled to public Internet 1454. Public Internet 1454 can be communicatively coupled to the NAT gateway 1438 contained in the control plane VCN 1416 and contained in the data plane VCN 1418. The service gateway 1436 contained in the control plane VCN 1416 and contained in the data plane VCN 1418 can be communicatively coupled to cloud services 1456.

In some embodiments, the data plane VCN 1418 can be integrated with customer tenancies 1470. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1446. Code to run the function may be executed in the VMs 1466(1)-(N), and the code may not be configured to run anywhere else on the data plane VCN 1418. Each VM 1466(1)-(N) may be connected to one customer tenancy 1470. Respective containers 1471(1)-(N) contained in the VMs 1466(1)-(N) may be configured to run the code. In this case, there can be a dual isolation (e.g., the containers 1471(1)-(N) running code, where the containers 1471(1)-(N) may be contained in at least the VM 1466(1)-(N) that are contained in the untrusted app subnet(s) 1462), which may help prevent incorrect or otherwise undesirable code from damaging the network of the IaaS provider or from damaging a network of a different customer. The containers 1471(1)-(N) may be communicatively coupled to the customer tenancy 1470 and may be configured to transmit or receive data from the customer tenancy 1470. The containers 1471(1)-(N) may not be configured to transmit or receive data from any other entity in the data plane VCN 1418. Upon completion of running the code, the IaaS provider may kill or otherwise dispose of the containers 1471(1)-(N).

In some embodiments, the trusted app subnet(s) 1460 may run code that may be owned or operated by the IaaS provider. In this embodiment, the trusted app subnet(s) 1460 may be communicatively coupled to the DB subnet(s) 1430 and be configured to execute CRUD operations in the DB subnet(s) 1430. The untrusted app subnet(s) 1462 may be communicatively coupled to the DB subnet(s) 1430, but in this embodiment, the untrusted app subnet(s) may be configured to execute read operations in the DB subnet(s) 1430. The containers 1471(1)-(N) that can be contained in the VM 1466(1)-(N) of each customer and that may run code from the customer may not be communicatively coupled with the DB subnet(s) 1430.

In other embodiments, the control plane VCN 1416 and the data plane VCN 1418 may not be directly communicatively coupled. In this embodiment, there may be no direct communication between the control plane VCN 1416 and the data plane VCN 1418. However, communication can occur indirectly through at least one method. An LPG 1410 may be established by the IaaS provider that can facilitate communication between the control plane VCN 1416 and the data plane VCN 1418. In another example, the control plane VCN 1416 or the data plane VCN 1418 can make a call to cloud services 1456 via the service gateway 1436. For example, a call to cloud services 1456 from the control plane VCN 1416 can include a request for a service that can communicate with the data plane VCN 1418.

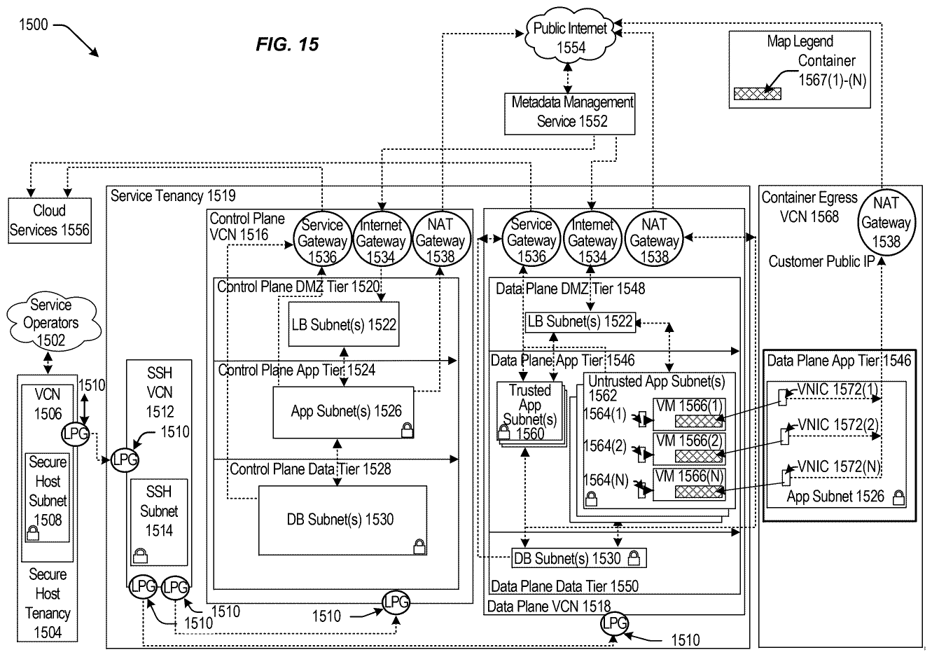

FIG. 15 is a block diagram 1500 illustrating another example pattern of an IaaS architecture, according to at least one embodiment. Service operators 1502 (e.g., service operators 1202 of FIG. 12) can be communicatively coupled to a secure host tenancy 1504 (e.g., the secure host tenancy 1204 of FIG. 12) that can include a virtual cloud network (VCN) 1506 (e.g., the VCN 1206 of FIG. 12) and a secure host subnet 1508 (e.g., the secure host subnet 1208 of FIG. 12). The VCN 1506 can include an LPG 1510 (e.g., the LPG 1210 of FIG. 12) that can be communicatively coupled to an SSH VCN 1512 (e.g., the SSH VCN 1212 of FIG. 12) via an LPG 1510 contained in the SSH VCN 1512. The SSH VCN 1512 can include an SSH subnet 1514 (e.g., the SSH subnet 1214 of FIG. 12), and the SSH VCN 1512 can be communicatively coupled to a control plane VCN 1516 (e.g., the control plane VCN 1216 of FIG. 12) via an LPG 1510 contained in the control plane VCN 1516 and to a data plane VCN 1518 (e.g., the data plane 1218 of FIG. 12) via an LPG 1510 contained in the data plane VCN 1518. The control plane VCN 1516 and the data plane VCN 1518 can be contained in a service tenancy 1519 (e.g., the service tenancy 1219 of FIG. 12).

The control plane VCN 1516 can include a control plane DMZ tier 1520 (e.g., the control plane DMZ tier 1220 of FIG. 12) that can include LB subnet(s) 1522 (e.g., LB subnet(s) 1222 of FIG. 12), a control plane app tier 1524 (e.g., the control plane app tier 1224 of FIG. 12) that can include app subnet(s) 1526 (e.g., app subnet(s) 1226 of FIG. 12), a control plane data tier 1528 (e.g., the control plane data tier 1228 of FIG. 12) that can include DB subnet(s) 1530 (e.g., DB subnet(s) 1430 of FIG. 14). The LB subnet(s) 1522 contained in the control plane DMZ tier 1520 can be communicatively coupled to the app subnet(s) 1526 contained in the control plane app tier 1524 and to an Internet gateway 1534 (e.g., the Internet gateway 1234 of FIG. 12) that can be contained in the control plane VCN 1516, and the app subnet(s) 1526 can be communicatively coupled to the DB subnet(s) 1530 contained in the control plane data tier 1528 and to a service gateway 1536 (e.g., the service gateway of FIG. 12) and a network address translation (NAT) gateway 1538 (e.g., the NAT gateway 1238 of FIG. 12). The control plane VCN 1516 can include the service gateway 1536 and the NAT gateway 1538.

The data plane VCN 1518 can include a data plane app tier 1546 (e.g., the data plane app tier 1246 of FIG. 12), a data plane DMZ tier 1548 (e.g., the data plane DMZ tier 1248 of FIG. 12), and a data plane data tier 1550 (e.g., the data plane data tier 1250 of FIG. 12). The data plane DMZ tier 1548 can include LB subnet(s) 1522 that can be communicatively coupled to trusted app subnet(s) 1560 (e.g., trusted app subnet(s) 1460 of FIG. 14) and untrusted app subnet(s) 1562 (e.g., untrusted app subnet(s) 1462 of FIG. 14) of the data plane app tier 1546 and the Internet gateway 1534 contained in the data plane VCN 1518. The trusted app subnet(s) 1560 can be communicatively coupled to the service gateway 1536 contained in the data plane VCN 1518, the NAT gateway 1538 contained in the data plane VCN 1518, and DB subnet(s) 1530 contained in the data plane data tier 1550. The untrusted app subnet(s) 1562 can be communicatively coupled to the service gateway 1536 contained in the data plane VCN 1518 and DB subnet(s) 1530 contained in the data plane data tier 1550. The data plane data tier 1550 can include DB subnet(s) 1530 that can be communicatively coupled to the service gateway 1536 contained in the data plane VCN 1518.

The untrusted app subnet(s) 1562 can include primary VNICs 1564(1)-(N) that can be communicatively coupled to tenant virtual machines (VMs) 1566(1)-(N) residing within the untrusted app subnet(s) 1562. Each tenant VM 1566(1)-(N) can run code in a respective container 1567(1)-(N), and be communicatively coupled to an app subnet 1526 that can be contained in a data plane app tier 1546 that can be contained in a container egress VCN 1568. Respective secondary VNICs 1572(1)-(N) can facilitate communication between the untrusted app subnet(s) 1562 contained in the data plane VCN 1518 and the app subnet contained in the container egress VCN 1568. The container egress VCN can include a NAT gateway 1538 that can be communicatively coupled to public Internet 1554 (e.g., public Internet 1254 of FIG. 12).

The Internet gateway 1534 contained in the control plane VCN 1516 and contained in the data plane VCN 1518 can be communicatively coupled to a metadata management service 1552 (e.g., the metadata management system 1252 of FIG. 12) that can be communicatively coupled to public Internet 1554. Public Internet 1554 can be communicatively coupled to the NAT gateway 1538 contained in the control plane VCN 1516 and contained in the data plane VCN 1518. The service gateway 1536 contained in the control plane VCN 1516 and contained in the data plane VCN 1518 can be communicatively coupled to cloud services 1556.

In some examples, the pattern illustrated by the architecture of block diagram 1500 of FIG. 15 may be considered an exception to the pattern illustrated by the architecture of block diagram 1400 of FIG. 14 and may be desirable for a customer of the IaaS provider if the IaaS provider cannot directly communicate with the customer (e.g., a disconnected region). The respective containers 1567(1)-(N) that are contained in the VMs 1566(1)-(N) for each customer can be accessed in real-time by the customer. The containers 1567(1)-(N) may be configured to make calls to respective secondary VNICs 1572(1)-(N) contained in app subnet(s) 1526 of the data plane app tier 1546 that can be contained in the container egress VCN 1568. The secondary VNICs 1572(1)-(N) can transmit the calls to the NAT gateway 1538 that may transmit the calls to public Internet 1554. In this example, the containers 1567(1)-(N) that can be accessed in real-time by the customer can be isolated from the control plane VCN 1516 and can be isolated from other entities contained in the data plane VCN 1518. The containers 1567(1)-(N) may also be isolated from resources from other customers.

In other examples, the customer can use the containers 1567(1)-(N) to call cloud services 1556. In this example, the customer may run code in the containers 1567(1)-(N) that requests a service from cloud services 1556. The containers 1567(1)-(N) can transmit this request to the secondary VNICs 1572(1)-(N) that can transmit the request to the NAT gateway that can transmit the request to public Internet 1554. Public Internet 1554 can transmit the request to LB subnet(s) 1522 contained in the control plane VCN 1516 via the Internet gateway 1534. In response to determining the request is valid, the LB subnet(s) can transmit the request to app subnet(s) 1526 that can transmit the request to cloud services 1556 via the service gateway 1536.

It should be appreciated that IaaS architectures 1200, 1300, 1400, 1500 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

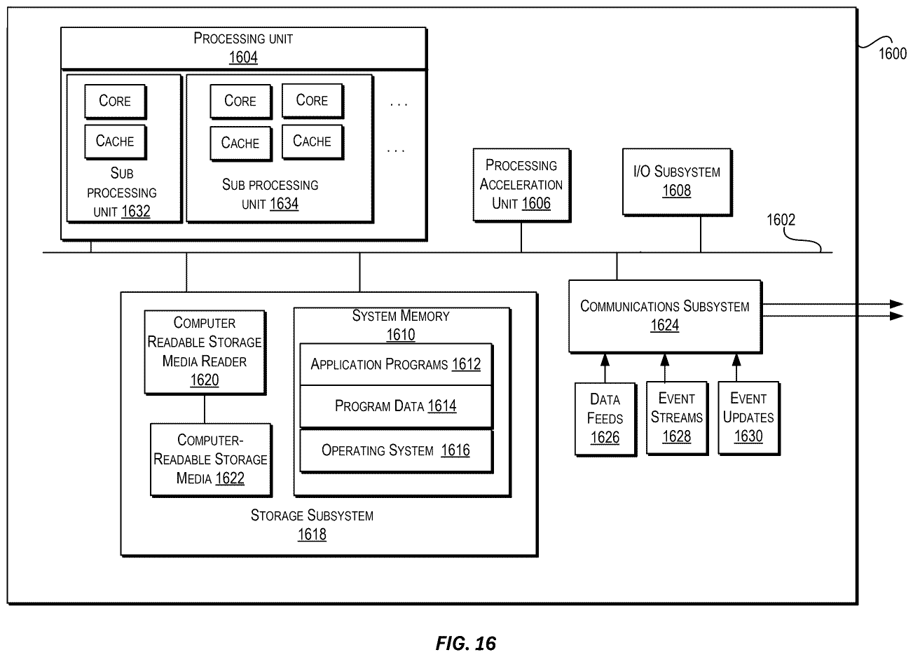

FIG. 16 illustrates an example computer system 1600, in which various embodiments may be implemented. The system 1600 may be used to implement any of the computer systems described above. As shown in the figure, computer system 1600 includes a processing unit 1604 that communicates with a number of peripheral subsystems via a bus subsystem 1602. These peripheral subsystems may include a processing acceleration unit 1606, an I/O subsystem 1608, a storage subsystem 1618 and a communications subsystem 1624. Storage subsystem 1618 includes tangible computer-readable storage media 1622 and a system memory 1610.

Bus subsystem 1602 provides a mechanism for letting the various components and subsystems of computer system 1600 communicate with each other as intended. Although bus subsystem 1602 is shown schematically as a single bus, alternative embodiments of the bus subsystem may utilize multiple buses. Bus subsystem 1602 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1604, which can be implemented as one or more integrated circuits (e.g., a conventional microprocessor or microcontroller), controls the operation of computer system 1600. One or more processors may be included in processing unit 1604. These processors may include single core or multicore processors. In certain embodiments, processing unit 1604 may be implemented as one or more independent processing units 1632 and/or 1634 with single or multicore processors included in each processing unit. In other embodiments, processing unit 1604 may also be implemented as a quad-core processing unit formed by integrating two dual-core processors into a single chip.

In various embodiments, processing unit 1604 can execute a variety of programs in response to program code and can maintain multiple concurrently executing programs or processes. At any given time, some or all of the program code to be executed can be resident in processor(s) 1604 and/or in storage subsystem 1618. Through suitable programming, processor(s) 1604 can provide various functionalities described above. Computer system 1600 may additionally include a processing acceleration unit 1606, which can include a digital signal processor (DSP), a special-purpose processor, and/or the like.

I/O subsystem 1608 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1600 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1600 may comprise a storage subsystem 1618 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1604 provide the functionality described above. Storage subsystem 1618 may also provide a repository for storing data used in accordance with the present disclosure.

As depicted in the example in FIG. 16, storage subsystem 1618 can include various components including a system memory 1610, computer-readable storage media 1622, and a computer readable storage media reader 1620. System memory 1610 may store program instructions that are loadable and executable by processing unit 1604. System memory 1610 may also store data that is used during the execution of the instructions and/or data that is generated during the execution of the program instructions. Various different kinds of programs may be loaded into system memory 1610 including but not limited to client applications, Web browsers, mid-tier applications, relational database management systems (RDBMS), virtual machines, containers, etc.

System memory 1610 may also store an operating system 1616. Examples of operating system 1616 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1600 executes one or more virtual machines, the virtual machines along with their guest operating systems (GOSs) may be loaded into system memory 1610 and executed by one or more processors or cores of processing unit 1604.

System memory 1610 can come in different configurations depending upon the type of computer system 1600. For example, system memory 1610 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1610 may include a basic input/output system (BIOS) containing basic routines that help to transfer information between elements within computer system 1600, such as during start-up.

Computer-readable storage media 1622 may represent remote, local, fixed, and/or removable storage devices plus storage media for temporarily and/or more permanently containing, storing, computer-readable information for use by computer system 1600 including instructions executable by processing unit 1604 of computer system 1600.

Computer-readable storage media 1622 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1622 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1622 may include, but is not limited to, Zip® drives, flash memory cards, universal serial bus (USB) flash drives, secure digital (SD) cards, DVD disks, digital video tape, and the like. Computer-readable storage media 1622 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1600.

Machine-readable instructions executable by one or more processors or cores of processing unit 1604 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1624 provides an interface to other computer systems and networks. Communications subsystem 1624 serves as an interface for receiving data from and transmitting data to other systems from computer system 1600. For example, communications subsystem 1624 may enable computer system 1600 to connect to one or more devices via the Internet. In some embodiments communications subsystem 1624 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 1624 can provide wired network connectivity (e.g., Ethernet) in addition to or instead of a wireless interface.

In some embodiments, communications subsystem 1624 may also receive input communication in the form of structured and/or unstructured data feeds 1626, event streams 1628, event updates 1630, and the like on behalf of one or more users who may use computer system 1600.

By way of example, communications subsystem 1624 may be configured to receive data feeds 1626 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1624 may also be configured to receive data in the form of continuous data streams, which may include event streams 1628 of real-time events and/or event updates 1630, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1624 may also be configured to output the structured and/or unstructured data feeds 1626, event streams 1628, event updates 1630, and the like to one or more databases that may be in communication with one or more streaming data source computers coupled to computer system 1600.

Computer system 1600 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1600 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening.

Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Various embodiments of this disclosure are described herein, including the best mode known for carrying out the disclosure. Variations of those embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
receiving a first request from a client system to provide a summary information for a patient of a healthcare provider, the summary information comprising patient history information describing at least one of a medical condition and a medical history of the patient;
determining at least one of a plurality of visit categories and a plurality of medical complaints for the patient;
retrieving data relevant to the first request from a plurality of sources based the at least one of the plurality of visit categories and the plurality of medical complaints;
generating a first input for a generative machine learning model, the first input comprising a first prompt based on the first request;
generating, by the generative machine learning model, a first query result from the first input, the first query result comprising a narrative section and a structured section extracted from one or more portions of the data;
generating, by the generative machine learning model, at least one suggested follow-on query selectable as a further request based on the first query result;
formatting the first query result and the at least one suggested follow-on query into a first brief summary; and
providing the first brief summary to a user interface at the client system.

2. The computer-implemented method of claim 1, wherein retrieving the data includes
accessing an electronic health record (EHR) to retrieve records including at least one of
medical history,
records from prior medical visits,
laboratory results,
diagnostic results,
past abnormal observation,
previously noted chief complaints,
known allergies,
medication history,
immunization records,
insurance information,
social history,
family history,
previous recommendations,
past and future appointments, and
messages sent through the EHR.

3. The computer-implemented method of claim 2, wherein accessing the EHR includes limiting a range of accessed records to items created within a predetermined time period prior to receiving the first request.

4. The computer-implemented method of claim 2, further comprising updating the data relevant to the first request based on updated information received from the patient at a visit to generate updated data, the updated information including at least one of vitals, one or more chief complaints for the visit, and intake notes at the visit.

5. The computer-implemented method of claim 4, further comprising:
receiving a second request from the client system based on the first brief summary;
retrieving data relevant to the second request, the data relevant to the second request including the updated data;
generating a second input for the generative machine learning model, the second input comprising a second prompt and one or more portions of the data relevant to the second request;
generating, by the generative machine learning model, a second query result from the second input based on the data relevant to the second request;
formatting the second query result into a second brief summary; and
providing the second brief summary to the user interface at the client system.

6. The computer-implemented method of claim 1, wherein
the narrative section includes a first selection from the first query result arranged into natural language text, and
the structured section includes a second selection from the first query result used to populate a plurality of preset windows within the first brief summary.

7. The computer-implemented method of claim 6, wherein at least one of the narrative section and the structured section includes one of a Uniform Resource Locator (URL) and a hovering window to enable display of additional information regarding a specific portion of the first brief summary.

8. A system comprising:
a computer system comprising one or more processors and memory storing computer executable instructions that, when executed by the one or more processors, configure the computer system to at least:
receiving a first request from a client system to provide a summary information for a patient of a healthcare provider for a visit, the summary information comprising patient history information describing at least one of a medical condition and a medical history of the patient;
determining at least one of a category of visit and a plurality of medical complaints for the patient, the category of visit including acute, wellness, follow-up, and generic;
obtain information regarding a chief complaint for the visit;
retrieving data relevant to the first request from a plurality of sources based on the at least one of the category of visit and the plurality of medical complaints;
formatting the data into intermediate representation (IR);
storing the IR at the computer system;
generating a first input for a generative machine learning model, the first input comprising a first prompt based on the first request;
generating, by the generative machine learning model, a first query result for the first input, the first query result comprising a narrative section and a structured section extracted from one or more portions of the data;
generating, by the generative machine learning model, at least one suggested follow-on query selectable as a further request;
formatting the first query result and the suggested follow-on query into a first brief summary; and
providing the first brief summary to a user interface at the client system.

9. The system of claim 8, wherein retrieving the data includes
accessing an electronic health record (EHR) to retrieve records including at least one of
medical history,
records from prior medical visits,
laboratory results,
diagnostic results,
past abnormal observation,
previously noted chief complaints,
known allergies,
medication history,
immunization records,
insurance information,
social history,
family history,
previous recommendations,
past and future appointments, and
messages sent through the EHR.

10. The system of claim 9, wherein accessing the EHR includes limiting a range of accessed records to items created within a predetermined time period prior to receiving the first request.

11. The system of claim 9, the computer system being further configured to update the data relevant to the first request based on updated information received from the patient at a visit to generate updated data, the updated information including at least one of vitals, one or more chief complaints for the visit, and intake notes at the visit.

12. The system of claim 11, the computer system being further configured to:
receiving a second request from the client system based on the first brief summary;
retrieving data relevant to the second request, the data relevant to the second request including the updated data;
generating a second input for the generative machine learning model, the second input comprising a second prompt and one or more portions of the data relevant to the second request;
generating, by the generative machine learning model, a second query result from the second input based on the data relevant to the second request;
formatting the second query result into a second brief summary; and
providing the second brief summary to the user interface at the client system.

13. The system of claim 9, wherein the narrative section includes a first selection from the first query result arranged into natural language text, and
the structured section includes a second selection from the first query result used to populate a plurality of preset windows within the first brief summary.

14. The system of claim 13, wherein at least one of the narrative section and the structured section includes one of a Uniform Resource Locator (URL) and a hovering window to enable display of additional information regarding a specific portion of the first brief summary.

15. A non-transitory computer-readable medium storing computer-executable instructions that, when executed by one or more processors of a computer system, configure the computer system to at least:
receiving a first request from a client system to provide a summary for a patient of a healthcare provider for a visit, the summary comprising patient history information describing at least one of a medical condition and a medical history of the patient;
determining at least one of a category of visit and a plurality of medical complaints for the patient, the category of visit including acute, wellness, follow-up, and generic;
obtain information regarding a chief complaint for the visit;
retrieving data relevant to the first request from a plurality of sources based on the at least one of the category of visit and the plurality of medical complaints;
generating a first input for a generative machine learning model, the first input comprising a first prompt based on the first request;
generating, by the generative machine learning model, a first query result from the first input, the first query result including a narrative section and a structured section extracted from one or more portions of the data;
generating, by the generative machine learning model, at least one suggested follow-on query selectable as a further request;
formatting the first query result and the suggested follow-on query into a first brief summary; and
providing the first brief summary to a user interface at the client system.

16. The non-transitory computer-readable medium of claim 15, wherein retrieving the data includes
accessing an electronic health record (EHR) to retrieve records including at least one of
medical history,
records from prior medical visits,
laboratory results,
diagnostic results,
past abnormal observation,
previously noted chief complaints,
known allergies,
medication history,
immunization records,
insurance information,
social history,
family history,
previous recommendations,
past and future appointments, and
messages sent through the EHR.

17. The non-transitory computer-readable medium of claim 16, wherein accessing the EHR includes limiting a range of accessed records to items created within a predetermined time period prior to receiving the first request.

18. The non-transitory computer-readable medium of claim 15, further comprising updating the data relevant to the first request based on updated information received from the patient at a visit, the updated information including at least one of vitals, one or more chief complaints for the visit, and intake notes at the visit.

19. The non-transitory computer-readable medium of claim 18, further comprising:
receiving a second request from the client system based on the first brief summary;
retrieving data relevant to the second request, the data relevant to the second request including the data as updated;
generating a second input for the generative machine learning model, the second input comprising a second prompt and one or more portions of the data relevant to the second request;
generating, by the generative machine learning model, a second query result from the second input based on the data relevant to the second request;
formatting the second query result into a second brief summary; and
providing the second brief summary to the user interface at the client system.

20. The non-transitory computer-readable medium of claim 15, wherein
the narrative section includes a first selection from the first query result arranged into natural language text, and
the structured section includes a second selection from the first query result used to populate a plurality of preset windows within the first brief summary.
